(19) United States
(12) Patent Application Publication
(10) Pub. No.: US 2025/0147047 A1
(43) Pub. Date: May 8, 2025

(54) Methods and Applications of Analyzing the Perfusate of an Ex Situ Perfused Kidney

(71) Applicants: VIB VZW, Gent (BE); KATHOLIEKE UNIVERSITEIT LEUVEN, Leuven (BE)

(72) Inventors: Bart Ghesquière, Leefdaal (BE); Ina Jochmans, Leuven (BE)

(21) Appl. No.: 18/835,535

(22) PCT Filed: Feb. 3, 2023
(86) PCT No.: PCT/EP2023/052628

§ 371 Date: Aug. 2, 2024

(30) Foreign Application Priority Data
Feb. 4, 2022 (EP) 22155190.6

(51) Int. Cl. G01N 33/68; G01N 33/50

(52) U.S. Cl. CPC G01N 33/6893; G01N 33/5082; G01N 2500/10; G01N 2800/347; G01N 2800/7019

(57) ABSTRACT

The invention relates to the field of analyzing parameters in the perfusate of an organ under ex situ organ perfusion conditions. In particular the identified parameters include 1 or more of arginine, glutamate, glucose, glutamine, lactate, alanine, leucine and isoleucine; and are capable of accurately determining the extent of damage present in the perfused organ. Several applications of the assessment of these identified parameters are also part of the invention.

### FIELD OF THE INVENTION

The invention relates to the field of analyzing parameters in the perfusate of an organ under ex situ organ perfusion conditions. In particular the identified parameters include 1 or more of arginine, glutamate, glucose, glutamine, lactate, alanine, leucine and isoleucine; and are capable of accurately determining the extent of damage present in the perfused organ. Several applications of the assessment of these identified parameters are also part of the invention.

### BACKGROUND OF THE INVENTION

Organ transplantation is the only life-saving treatment for end-stage organ failure. While over 130,000 organs are transplanted every year, the World Health Organization estimates 10 times as many organs are needed (https://www.who.int/transplantation/donation/taskforce-transplantation/en/). For kidney transplantation, for instance, a staggering 15 to 20% of kidney grafts offered are never transplanted while patients die on the waiting list. Organ shortage led to expansion of the donor pool. For kidneys for instance, expanded criteria donors (ECDs) include donors with defined comorbidities and those donated after circulatory death. A major reason for kidney graft discard is the fear of early graft failure associated with “poor organ quality”. However, tools to assess organ quality are not very accurate and a substantial number of discarded kidneys is estimated to nevertheless provide life-sustaining function. The challenge in organ transplantation is therefore to accurately predict the future function of the donor organ before it is transplanted.

In recent years, there has been a renewed interest and application of organ storage using ex-vivo machine perfusion (MP; hypothermic or normothermic) conditions (as opposed to traditional static cold storage (SCS) of a donor organ). MP is used for donor kidneys, livers, lungs and hearts. Reportedly, emerging evidence suggests that MP increases the number of donor organs that can be transplanted and would otherwise have been discarded (e.g. Watson et al. 2018, Am J Transplant 1-16; Nasralla et al. 2018, Nature 557:50-56; Cannon et al. 2016, Transplant Res Risk Management 8:1-7; Bellini et al. 2019, J Clin Med 8:1221; Hosgood et al. 2011, Transplantation 92:735-738; Hosgood et al. 2014, Am J Transplant 14:490-491; Hosgood et al. 2018, Br J Surg 105:388-394; Mergental et al. 2020, Nat Commun 11:2939). Besides increasing preservation of a donor organ, MP also holds promise for donor organs to be treated or repaired before transplantation; for instance lungs of patient died from pulmonary embolism were during ex-vivo MP treated with clot lysing agent and successfully transplanted (Senior 2018, Nat Biotech 36:488), or a donor organ can be treated ex vivo with an enzyme to temporarily ablate MHC class I antigens as a way to dampen transplant rejection reactions (e.g. WO 00/48462), or with alpha 1-antitrypsin to dampen ischemic reperfusion injury (e.g. WO 2020/026227).

Assessment of donor organs prior to grafting in the recipient currently usually includes a mix of donor-related criteria and basic visual, olfactory and tactile methods, is subjective and non-standardized (Senior 2018, Nat Biotech 36:488). Such approach does not allow for reliable quantification of organ damage or prediction of future organ function.

Such assessment of donor organs may further include analysis of a procurement biopsy and assessment during hypothermic machine perfusion, but this still leads to discard of donor organs potentially useable for transplantation (Kabagambe et al. 2019, Transplantation 103: 392-400).

Donor organ damage can occur due to warm ischemia (WI; for instance for organ retrieved from donor after circulatory death) or due to cold ischemia (CI; for instance for organ stored on ice). Ischemic or hypoxic damage is further determined by the ischemic period(s), such as e.g. influenced by the distance between the organ retrieval center and the organ recipient center. Such periods of ischemia result, upon reperfusion, in ischemia reperfusion injury (IRI). IRI can manifest itself clinically as acute injury (e.g. acute kidney injury), delayed graft function, or primary non function. Duration of cold and/or warm ischemia period(s) is another factor that is usually included in the assessment of the fitness for transplantation of a donor organ. Invasive ischemia-related DNA methylation markers have been described (WO2019122303A1), but reliable non-invasive markers objectively informing on ischemic damage of an organ as occurred in the pre-transplant period are currently not available.

Often used scoring systems to assess kidney graft failure risk in view of deceased donor characteristics are the Kidney Donor Risk Index or KDRI, and the Kidney Donor Profile Index (KDPI; a percentile version of the KDRI) (Zhong et al. 2019, Transplantation 103:1714-1721).

Bellini et al. 2021 (Int J Med Sci 22:1121) lists in Table 1 viability assessment parameters during ex vivo MP of kidneys. These parameters can be more or less mechanistically grouped: (i) macroscopic appearance, renal blood flow and urine output; (ii) pressure, flow and resistance readings during MP; (iii) glucose consumption; (iv) oxygen consumption; (v) final glycolysis products; (vi) ATP depletion or ATP/ADP ratio; (vii) biomarkers of cellular injury; (viii) inline microdialysis; (ix) mRNA profiling; and (x) flavin mononucleotide levels.

In summarizing the advances in kidney transplantation, Abramowicz et al. 2018 (Nephrol Dial Transplant 33:1699-1707) indicated that, in relation to kidney MP, perfusion parameters (flow and resistance) and injury markers determined in the perfusate (neutrophil gelatinase associated lipocalin [NGAL], interleukin 18, liver-type fatty acid binding protein [FABP]) poorly correlate with kidney allograft function.

Whereas switching to ex-vivo perfusion allows for sampling of the perfusate (and in case of kidneys possibly also of urine, in case of livers possibly also of bile), reliable metrics to determine whether a donor organ (e.g. kidney) is viable and safe to transplant are currently not available (Hamelink et al., Transplantation, doi: 10.1097/TP.0000000000003817.—listing in Table 2 a number of potential biomarkers; De Beule & Jochmans 2020, J Clin Med 9:879). According to DiRito et al. 2021 (Am J Transplant 21:161-173), thousands of kidneys from higher-risk donors are discarded annually because of the increased likelihood of complications posttransplant. Given the severe organ shortage, there is a critical need to improve utilization of these organs.

### SUMMARY OF THE INVENTION

In a general aspect, the invention relates to methods of analyzing the perfusate of a kidney which is ex vivo or ex situ perfused, comprising:
sampling the perfusate during or after initiating the perfusion;
determining the presence, level or concentration of 1 or more analytes in the perfusate sample(s), wherein the analyte is arginine, or wherein the analytes are arginine and 1 or more of glutamate, glutamine, glucose, lactate, alanine, leucine and/or isoleucine.

Such methods in particularly can be methods of determining the presence, absence or severity of ischemic damage in a kidney and is further comprising:
determining ischemic damage to be present in the perfused kidney when the determined presence, level or concentration of the 1 or more analytes is deviating or is significantly deviating from the control or reference level or concentration of the 1 or more analytes; or, alternatively, determining ischemic damage to be absent in the perfused kidney when the determined level or concentration of the 1 or more analytes is not or not significantly deviating from the control or reference level or concentration of the 1 or more analytes; or, alternatively, determining the severity of ischemic damage in the perfused organ from the extent of deviation of the determined level or concentration of the 1 or more analytes from the control or reference level or concentration of the 1 or more analytes.

Further methods of the invention are methods of therapeutic optimization of, repair of, or reconditioning of a kidney prior to transplantation, comprising one or more steps of:
connecting the kidney to an ex vivo or ex situ perfusion device;
performing the therapeutic optimization, repair or reconditioning of the perfused kidney during perfusion;
sampling the perfusate during or after initiating the perfusion;
determining the presence, level or concentration of 1 or more analytes in the perfusate sample(s), wherein the analyte is arginine, or wherein the analytes are arginine and 1 or more of glutamate, glutamine, glucose, lactate, alanine, leucine and/or isoleucine;

Further methods of the invention are methods of developing or optimizing kidney perfusion solutions or kidney preservation solutions, or of optimizing kidney perfusion conditions, comprising:
connecting the kidney to an ex vivo or ex situ perfusion device;
perfusing the kidney with a test perfusion or preservation solution, or perfusing the kidney under test perfusion conditions;
monitoring the presence, level or concentration of 1 or more analytes in the perfusate, wherein the analyte is arginine, or wherein the analytes are arginine and 1 or more of glutamate, glutamine, glucose, lactate, alanine, leucine and/or isoleucine during perfusion.

Further methods of the invention are methods of screening for modalities capable of reversing or partially reversing defects of an kidney; or of screening for modalities capable of reversing or partially reversing ischemic damage in a kidney; or of screening for modalities capable of improving or enhancing kidney function, comprising one or more steps of:
connecting the kidney to an ex vivo or ex situ perfusion device;
adding to the perfusion solution a test modality, wherein the test modality is designed to reverse or to partially reverse a defect of or in a kidney; or wherein the test modality is designed to reverse or to partially reverse ischemic damage in a kidney; or wherein the test modality is designed to improve or enhance kidney function;
monitoring the presence, level or concentration of 1 or more analytes in the perfusate, wherein the analyte is arginine, or wherein the analytes are arginine and 1 or more of glutamate, glutamine, glucose, lactate, alanine, leucine and/or isoleucine.

In particular, in any of the foregoing methods, the analytes to be determined in the perfusate can be further defined:
when the presence, level or concentration of glutamate is determined, the presence, level or concentration of at least 1 or more of the analytes arginine, glutamine, glucose, lactate, alanine, leucine and/or isoleucine is determined; or
when the presence, level or concentration of glucose is determined, the presence, level or concentration of at least 1 or more of the analytes arginine, glutamate, glutamine, alanine and/or isoleucine is determined; or
when the presence, level or concentration of lactate is determined, the presence, level or concentration of at least 1 or more of the analytes arginine, glutamate, glutamine, alanine, leucine and/or isoleucine is determined; or
when the presence, level or concentration of leucine is determined, the presence, level or concentration of at least 1 or more of the analytes arginine, glutamate, glutamine, lactate, alanine, and/or isoleucine is determined.

In particular, in any of the foregoing methods, the analyte to be determined in the perfusate can be defined as arginine.

In another aspect, the invention relates to a machine perfusion apparatus or device, an organ transporter, or an organ cassette, further characterized in that it is comprising a unit or module for determining the presence, level or concentration of 1 one or more analytes in the perfusate of an organ ex situ perfused with a perfusion solution, wherein the 1 or more analytes is arginine, or is arginine and 1 or more of glutamate, glutamine, glucose, lactate, alanine, leucine and/or isoleucine.

Further methods of the invention are methods of analyzing the perfusate of an organ perfused ex vivo or ex situ with a perfusion solution, comprising:
sampling the perfusate at different time points during the ex vivo or ex situ perfusion;
determining a reference time point which is the time point during perfusion at which the organ wash-out fraction has homogenized with the initial perfusion solution; and, optionally,
setting the presence, level or concentration of an analyte of interest determined in the perfusate at the reference time point as reference or control level or concentration of the analyte of interest.

### BRIEF DESCRIPTION OF THE DRAWINGS

FIG. 1. Experimental setup: a pig kidney is coupled or connected to an ex-vivo perfusion apparatus and perfused with a perfusate or perfusion solution. At regular time-points, samples of the perfusate are collected for analysis. Control: non-ischemic conditions; CI: cold ischemic conditions; WI: warm ischemic conditions; HE: heat exchanger; O2: gas exchanger.

FIG. 2. Heat map representing relative changes of analytes (listed on the right) in the perfusate of perfused kidneys over time (as indicated in second top row in minutes). The relative changes of analytes are the changes relative to the average concentration of an analyte in the perfusate independent of time or condition. Control: non-ischemic conditions; CI: cold ischemic conditions; WI: warm ischemic conditions. 2A: red blood cell (RBC)-based perfusion solution; 2B: whole blood based perfusion solution.

FIG. 3. Analysis of changes over time (X-axis, minutes; versus the reference point determined at 15 min after start of the perfusion) in the amounts of glutamate (A), arginine (B) and hexose (glucose) (C) in the perfusate of pig kidneys perfused ex-vivo with a red blood cell (RBC)-based perfusate. Left data point of each X-axis point: non-ischemic conditions; middle data point of each X-axis point: cold ischemic conditions; right data point of each X-axis point: warm ischemic conditions. Y-axis value of 100%: no change in amount of analyte, or uptake and secretion of the analyte by the perfused kidney are balanced; Y-axis value above 100%: net secretion of the analyte by the perfused kidney; Y-axis below above 100%: net uptake of the analyte by the perfused kidney. As reference point, the composition of the perfusate 15 minutes after start of the perfusion was taken.

FIG. 4. Analysis of changes over time (X-axis, minutes; versus the reference point determined at 15 min after start of the perfusion) in the amounts of glutamate (A), arginine (B) and hexose (glucose) (C) in the perfusate of pig kidneys perfused ex-vivo with a whole blood based perfusate. Left data point of each X-axis point: non-ischemic conditions; middle data point of each X-axis point: cold ischemic conditions; right data point of each X-axis point: warm ischemic conditions. Y-axis value of 100% uptake and secretion of the analyte by the perfused kidney are balanced; Y-axis value above 100%: net secretion of the analyte by the perfused kidney; Y-axis below above 100%: net uptake of the analyte by the perfused kidney. As reference point, the composition of the perfusate 15 minutes after start of the perfusion was taken.

FIG. 5. Spaghetti plot of fold changes of arginine concentrations (μMol) during 2 hours of normothermic perfusion (corrected for time 0) of human kidneys. Individual human kidneys are indicated by a number on top of each of the plot curves. For kidneys 1, 2, and 6, arginine concentration always was below the detection limit (FC=1).

FIG. 6. Scatter plot of human kidney function (eGFR) at 3 months (“eGFR 3 months”) and the relative change of arginine over 2 hours kidney perfusion (“FC—Arginine 2 hrs”). Arrow on eGFR axis: ˜mean eGFR value. Individual human kidneys are indicated by the same numbering as in FIG. 5.

### DETAILED DESCRIPTION

In work leading to the current invention, normal, cold ischemically, and warm ischemically treated/damaged pig kidneys were subjected to ex vivo or ex situ (used interchangeably herein) machine perfusion (MP) with a perfusion solution. By monitoring changes of metabolites in the perfusate over time, easily measurable biochemical perfusate markers were identified capable of robustly identifying whether a perfused kidney was injured or damaged by cold or warm ischemia. Furthermore, these markers are indicators of (future) kidney function independent of their condition (as measured by clearance of creatinine). Preliminary data collected from analyses on perfusate of human kidneys indicate that, although conditions were not fully comparable with the controlled perfusion of the pig kidneys, a drop in arginine (one of the perfusate markers identified during in the pig kidney perfusion experiments) was observed during perfusion, and a trend was emerging for the change in arginine levels during perfusion to be correlated with post-transplantation function of the human kidney.

Clearly, such biochemical perfusate markers can contribute to increasing the pool of organs available for transplantation, an unmet need increasing in amplitude as e.g. the overall number of donor kidneys within Eurotransplant has decreased despite of an increasing demand (Abramowicz et al. 2018, Nephrol Dial Transplant 33:1699-1707). The herein identified biochemical perfusate markers provide objective tools to assess the quality and (future) function of donor kidneys, in particular to assess the quality and (future) function of donor kidneys that would otherwise be discarded for transplantation, as such capable of identifying donor kidneys with a favorable risk-benefit ratio and therewith increasing the pool of kidneys available for transplantation.

Furthermore, the herein identified biochemical perfusate markers indicative of organ damage/injury (such as ischemic injury), organ quality and (future) function can be applied in many different methods. The monitoring of the herein identified biochemical perfusate markers indicative of organ damage/injury, organ quality and (future) function can be used in methods of developing or optimizing organ perfusion solutions or organ preservation solutions.

The monitoring of the herein identified biochemical perfusate markers indicative of organ damage/injury, organ quality and (future) function can be used in methods of screening for modalities capable of reversing or partially reversing ischemic damage in the organ, or in methods of screening for modalities capable of reversing or partially reversing other defects of the organ, or in methods of screening for modalities capable of improving or enhancing organ function. With modality is meant any compound of any nature. Modalities include small molecules, peptides, proteins, nucleic acids, antibodies, metabolites, probiotics, etc. and combinations of any of these (e.g. peptide, protein or antibody carrying a small molecule payload).

Likewise, the monitoring of the herein identified biochemical perfusate markers indicative of damage/injury, organ quality and (future) function can also be used in methods of/for therapeutic optimization of, repair of, or reconditioning the organ prior to transplantation, such as to minimize delayed graft function, acute rejection or ischemic reperfusion injury. Such therapeutic optimization, repair or reconditioning includes ex-vivo surgery (e.g. removal of necrotic or cancerous tissue), ex-vivo radiation or ex-vivo chemotherapy (preventing other parts of the recipient's body to be exposed to radiation or chemotherapy). Such therapeutic optimization, repair or reconditioning further includes immunomodulation or immunomodification of the perfused organ such as to minimize acute or chronic rejection of an allograft by the recipient or recipient's body. Gene manipulation of at least the vascular endothelium during warm perfusion of kidneys has for instance been demonstrated (Brasile et al. 2002, Transplant Proc 35:2624). Further exemplified therapeutic interventions during hypothermic or normothermic machine perfusion of kidneys include cellular therapies (e.g. with mesenchymal stromal cells (MSCs) promoting regeneration and repair, and protecting against acute and chronic kidney injury; or with multipotent adult progenitor cells (MAPCs) improving tissue perfusion and lowering levels of tubular injury and inflammation), gene therapies (e.g. matrix metalloproteinase (MMP-2) siRNA; mixture of siRNAs targeting C3, Fas and RelB; caspase 3 siRNA; beta2 microglobulin shRNA and class II transactivator), biological therapies (e.g. Corline heparin conjugate (CHC); thrombalexin) and antibody therapies (e.g. CD47 blocking antibody)(summarized by Hosgood et al. 2021, Transpl Int 34:224-232—see Tables 1 and 2 therein). DiRito et al. 2021 (Am J Transplant 21:161-173) applied combined delivery of plasminogen and tissue plasminogen activator during normothermic machine perfusion of kidneys to remove microvascular obstructions/plugs which reduced markers of renal injury, improved markers of renal function, and improved delivery of vascular-targeted nanoparticles. Other examples of ex vivo donor organ treatment include treatment with an enzyme to temporarily ablate MHC class I antigens as a way to dampen transplant rejection reactions (e.g. WO 00/48462), or with alpha 1-antitrypsin to dampen ischemic reperfusion injury (e.g. WO 2020/026227). As outlined in e.g. WO2019122303A1 and WO2020254364A1, the methylation status of kidney DNA as determined at the time of transplantation is predictive for future, post-transplant allograft function, in particular for different types of future fibrosis; this opens the avenue for treating the allograft with e.g. DNA demethylating agents prior to transplantation.

In all these instances, organ damage/injury, organ quality and (future) function can be monitored by monitoring or measuring the herein identified biochemical perfusate markers during the ex vivo or ex situ perfusion period. In particular, in all these instances, organ function/injury, quality and (future) function can be monitored by monitoring or measuring the described biochemical perfusate markers indicative of ischemic organ damage during the ex vivo perfusion period. In one particular embodiment, the organ is an allograft or a xenograft. In another particular embodiment, the organ is an autograft removed from a subject's body and subjected to ex vivo therapeutic optimization, repair, or reconditioning prior to re-implantation in the subject's body. In particular, the organ is a kidney, more in particular a human kidney.

The invention in several aspects therefore relates to any one of several methods:
(i) methods of/for analyzing the perfusate of an organ which is ex vivo or ex situ machine perfused; or
(ii) methods of/for analyzing (the presence or absence of) ischemic damage in an organ which is ex vivo or ex situ machine perfused; or
(iii) methods of/for determining fitness, function or quality of an organ which is ex vivo or ex situ machine perfused (such as prior to potential grafting into a recipient); or
(iv) methods of/for prospectively determining the potential post-transplant or post-transplantation function of an organ which is ex vivo or ex situ machine perfused; or
(v) methods of/for monitoring, maintaining, and/or restoring viability or function of an organ which is ex vivo or ex situ machine perfused; or
(vi) methods of/for therapeutic optimization of, repair of, or reconditioning of an organ prior to transplantation (such as to minimize delayed graft function, acute rejection or ischemic reperfusion injury); or
(vii) methods of/for developing or optimizing organ perfusion solutions or organ preservation solutions, or of/for optimizing organ perfusion conditions; or
(viii) methods of screening for modalities capable of reversing or partially reversing defects of an organ; or
(ix) methods of/for screening for modalities capable of reversing or partially reversing the ischemic damage in an organ; or
(x) methods of screening for modalities capable of improving or enhancing organ function.

In particular, all of these methods include a step of determining, measuring, assessing, analyzing or monitoring the level or concentration of 1 or more analytes in the perfusate sample(s), wherein the analytes are arginine, glutamate, glutamine, glucose, lactate, alanine, leucine and/or isoleucine.

In particular, in all of these methods, the organ is a kidney, even more in particular a kidney of a mammalian species, more in particular a human kidney.

These methods are described in more detail hereinafter.

In particular, methods of/for analyzing the perfusate of an organ which is ex vivo or ex situ (machine) perfused are comprising one or more steps of:
sampling the perfusate during or after initiating the (ex vivo or ex situ/machine) perfusion;
determining, measuring, assessing, analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

Further in particular, the methods of/for determining (the presence or absence of, severity of) ischemic damage in an organ which is ex vivo or ex situ (machine) perfused are comprising one or more steps of:
sampling the perfusate during or after initiating the (ex vivo or ex situ/machine) perfusion;
determining, measuring, assessing, analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
determining ischemic damage to be present in the perfused organ when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ); or, alternatively, determining ischemic damage to be absent in the perfused organ when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ); or, alternatively, determining the severity of ischemic damage in the perfused organ from the (extent of) deviation of the determined, measured, assessed or analyzed presence, level or concentration of the 1 or more analytes from the control or reference level or concentration of the 1 or more analytes (such as representative for a healthy (ex vivo or ex situ/machine) perfused organ).

As it is can be easily envisaged that deviation of the presence, level or concentration of the 1 or more analytes from the control level or concentration of the 1 or more analytes is correlated with the extent of organ damage, severity of ischemic damage can indeed be derived from the extent of deviation of the presence, level or concentration of the 1 or more analytes from the control level or concentration of the 1 or more analytes.

Further in particular, the methods of/for determining fitness, function or quality of an organ which is ex vivo or ex situ (machine) perfused (such as prior to potential grafting into a recipient) are comprising one or more steps of:
sampling the perfusate during or after initiating the (ex vivo or ex situ/machine) perfusion;
determining, measuring, assessing, analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
determining the perfused organ to be fit, optimally functioning or of good quality when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ); or, alternatively, determining the perfused organ not to be fit, to be functioning sub-optimally or badly, or to be of sub-optimal, poor, or bad quality when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ).

An additional step could be comprised in these methods:
selecting a fit organ, an optimally functioning organ or an organ of good quality for subsequent grafting into a recipient.

Alternative methods of/for determining fitness, function or quality of an organ which is ex vivo or ex situ (machine) perfused (such as prior to potential grafting into a recipient) can be defined based on clinical observations with perfused human kidneys (see Example 3). Such alternative methods are comprising one or more steps of:
sampling the perfusate during or after initiating the (ex vivo or ex situ/machine) perfusion;
determining, measuring, assessing or analyzing the presence, level or concentration of the analyte arginine in the perfusate sample(s);
determining the perfused organ to be fit, optimally functioning or of good quality when the analyte arginine is not detectable, the analyte arginine is absent, or its presence cannot be determined in the perfusate sample(s).

In one embodiment such alternative methods are comprising one or more steps of:
subjecting the organ to ex vivo or ex situ (machine) perfusion wherein the perfusion comprises a period of hypothermic perfusion followed by a period of normothermic perfusion;
sampling the perfusate during or after initiating the (ex vivo or ex situ/machine) perfusion;
determining, measuring, assessing or analyzing the presence, level or concentration of the analyte arginine in the perfusate sample(s);
determining the perfused organ to be fit, optimally functioning or of good quality when the analyte arginine is not detectable, the analyte arginine is absent, or its presence cannot be determined in the perfusate sample(s) of the normothermic perfusion; or when the level or concentration of the analyte arginine is between two sampling times rapidly decreasing to non-detectable or non-determinable levels or concentrations as determined, measured, assessed or analyzed in the perfusate sample(s) of the normothermic perfusion.

In one further embodiment to these alternative methods, the analyte arginine in the samples of the normothermic perfusion phase is decreasing to non-detectable or non-determinable levels or concentrations in up to 30 min after start of the normothermic perfusion; in such situation the two sampling times are separated by up to 30 minutes.

An additional step could be comprised in these methods:
selecting a fit organ, an optimally functioning organ or an organ of good quality for subsequent grafting into a recipient.

Further in particular, methods of/for determining or of/for prospectively determining the potential post-transplant or post-transplantation function of an organ which is ex vivo or ex situ (machine) perfused are comprising one or more steps of:
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
determining or prospectively determining the potential post-transplant or post-transplantation function of the perfused organ to be sufficient, good or optimal when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ); or, alternatively, determining or prospectively determining the potential post-transplant or post-transplantation function of the perfused organ to be insufficient, sub-optimal, poor or bad when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ).

Alternative methods of/for determining or of/for prospectively determining the potential post-transplant or post-transplantation function of an organ which is ex vivo or ex situ (machine) perfused can be defined based on clinical observations with perfused human kidneys (see Example 3). Such methods are comprising one or more steps of:
sampling the perfusate during or after initiating the (ex vivo or ex situ/machine) perfusion;
determining, measuring, assessing or analyzing the presence, level or concentration of the analyte arginine in the perfusate sample(s);
determining or prospectively determining the potential post-transplant or post-transplantation function of the perfused organ to be sufficient, good or optimal when the determined, measured, assessed or analyzed presence, level or concentration of the analyte arginine is not detectable, the analyte arginine is absent, or its presence cannot be determined in the perfusate sample(s) of the normothermic perfusion.

In one embodiment such alternative methods are comprising one or more steps of:
subjecting the organ to ex vivo or ex situ (machine) perfusion wherein the perfusion comprises a period of hypothermic perfusion followed by a period of normothermic perfusion;
sampling the perfusate during or after initiating the (ex vivo or ex situ/machine) perfusion;
determining, measuring, assessing or analyzing the presence, level or concentration of the analyte arginine in the perfusate sample(s);
determining or prospectively determining the potential post-transplant or post-transplantation function of the perfused organ to be sufficient, good or optimal when the determined, measured, assessed or analyzed presence, level or concentration of the analyte arginine in the perfusate sample(s) of the normothermic perfusion phase is not detectable, the analyte arginine is absent, or its presence cannot be determined; or when the level or concentration of the analyte arginine is between two sampling times rapidly decreasing to non-detectable or non-determinable levels or concentrations as determined, measured, assessed or analyzed in the perfusate sample(s) of the normothermic perfusion.

In one further embodiment to these alternative methods, the analyte arginine in the samples of the normothermic perfusion phase is decreasing to non-detectable or non-determinable levels or concentrations in up to 30 min after start of the normothermic perfusion; in such situation the two sampling times are separated by up to 30 minutes.

In one further embodiment, these alternative methods of/for determining or of/for prospectively determining the potential post-transplant or post-transplantation function of an organ which is ex vivo or ex situ (machine) perfused are methods of/for determining or of/for prospectively determining the potential post-transplant or post-transplantation estimated glomerular filtration rate (eGFR) of a kidney which is ex vivo or ex situ (machine) perfused. More in particular the eGFR herein is the eGFR at 3 months post-transplant or post-transplantation.

Further in particular, the methods of/for monitoring, maintaining, and/or restoring viability or function of an organ which is ex vivo or ex situ (machine) perfused are comprising one or more steps of:
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
monitoring the viability or function of the perfused organ, wherein the perfused organ is determined to be viable, to remain viable, or to function properly when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ; or, alternatively, monitoring the viability or function of the perfused organ, wherein the viability or function of the perfused organ is determined to be impacted when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ); and/or
when the presence, level or concentration of the 1 or more analytes is deviating or is significantly deviating from the control or reference level or concentration of the 1 or more analytes (such as representative for a healthy (ex vivo or ex situ/machine) perfused organ): maintaining and/or restoring the viability or function of the perfused organ by adjusting the perfusion conditions and/or perfusion solution thereby maintaining and/or restoring the viability or function of the perfused organ.

Alternatively, such methods of/for maintaining and/or restoring viability or function of an organ which is ex vivo or ex situ (machine) perfused are comprising one or more steps of:
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
adjusting the perfusion conditions and/or perfusion solution when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ), thereby maintaining and/or restoring the viability or function of the perfused organ.

Further in particular, the methods of/for therapeutic optimization of, repair of, or reconditioning of an organ prior to transplantation (such as to minimize delayed graft function, acute rejection or ischemic reperfusion injury) are comprising one or more steps of:
ex vivo or ex situ coupling or connecting the organ to an ex vivo or ex situ (machine) perfusion device;
performing the therapeutic optimization, repair or reconditioning of the perfused organ during (ex vivo or ex situ/machine) perfusion;
monitoring the presence, level or concentration of 1 or more analytes in the perfusate, wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

The monitoring of the presence, level or concentration of the 1 or more analytes may include:
sampling the perfusate during or after initiating the (ex vivo or ex situ/machine) perfusion, or, alternatively, during or after the therapeutic optimization, repair or reconditioning of the perfused organ;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

Alternatively, such methods of/for therapeutic optimization of, repair of, or reconditioning of an organ prior to transplantation (such as to minimize delayed graft function, acute rejection or ischemic reperfusion injury) are comprising one or more steps of:
ex vivo or ex situ coupling or connecting the organ to an ex vivo or ex situ (machine) perfusion device;
performing the therapeutic optimization, repair or reconditioning of the perfused organ during (ex vivo or ex situ/machine) perfusion;
sampling the perfusate during or after initiating the (ex vivo or ex situ/machine) perfusion, or, alternatively, during or after the therapeutic optimization, repair or reconditioning of the perfused organ;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
monitoring the viability or function of the perfused organ during the therapeutic optimization, repair or reconditioning, wherein the perfused organ is determined to be viable, to remain viable, or to function properly when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ); or, alternatively, wherein the viability or function of the perfused organ is determined to be impacted when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ).

Further in particular, such methods of/for therapeutic optimization of, repair of, or reconditioning of an organ prior to transplantation (such as to minimize delayed graft function, acute rejection or ischemic reperfusion injury) are comprising one or more steps of:
performing ex vivo or ex situ therapeutic optimization, repair or reconditioning of the organ;
coupling or connecting the optimized, repaired or reconditioned organ to an ex vivo or ex situ (machine) perfusion device;
monitoring the presence, level or concentration of 1 or more analytes in the perfusate, wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

The monitoring of the presence, level or concentration of the 1 or more analytes may include:
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

Alternatively, such methods of/for therapeutic optimization of, repair of, or reconditioning of an organ prior to transplantation (such as to minimize delayed graft function, acute rejection or ischemic reperfusion injury) are comprising one or more steps of:
performing ex vivo or ex situ therapeutic optimization, repair or reconditioning of the organ;
coupling or connecting the optimized, repaired or reconditioned organ to an ex vivo or ex situ (machine) perfusion device;
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
monitoring the viability or function of the perfused organ, wherein the perfused organ is determined to be viable, to remain viable, or to function properly when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ); or, alternatively, wherein the viability or function of the perfused organ is determined to be impacted when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ).

Further in particular, the methods of/for developing or optimizing organ perfusion solutions or organ preservation solutions, or of/for optimizing organ perfusion conditions, are comprising one or more steps of:
coupling or connecting the organ to an ex vivo or ex situ machine perfusion device;
perfusing the organ with a test perfusion or preservation solution, or perfusing the organ under test perfusion conditions;
monitoring the presence, level or concentration of 1 or more analytes in the perfusate, wherein the analytes are arginine, glutamate, glutamine, glucose, lactate, alanine, leucine and/or isoleucine during perfusion; or wherein the analyte is arginine; or wherein the analytes are arginine and 1 or more of, glutamate, glutamine, glucose, lactate, alanine, leucine and/or isoleucine.

The monitoring of the presence, level or concentration of the 1 or more analytes may include:
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

Alternatively, such methods of/for developing or optimizing organ perfusion solutions or organ preservation solutions, or of/for optimizing perfusion conditions, are comprising one or more steps of:
coupling or connecting the organ to an ex vivo or ex situ (machine) perfusion device;
perfusing the organ with a test perfusion or preservation solution, or perfusing the organ under test perfusion conditions;
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
monitoring the viability or function of the perfused organ, wherein the perfused organ is determined to be viable, to remain viable, or to function properly when the presence, level or concentration of the 1 or more analytes is not or not significantly deviating from the control or reference level or concentration of the 1 or more analytes (such as representative for a healthy (ex vivo or ex situ/machine) perfused organ); or, alternatively, wherein the viability or function of the perfused organ is determined to be impacted when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ).

An additional step could be comprised in these methods:
selecting a test perfusion or preservation solution, or selecting test perfusion conditions which is/are not or not significantly affecting or impacting the viability or function of the perfused organ; or optimizing a test perfusion or preservation solution, or optimizing test perfusion conditions which is/are affecting or impacting the viability or function of the perfused organ.

Further in particular, the methods of screening for modalities capable of reversing or partially reversing defects of an organ; or the methods of/for screening for modalities capable of reversing or partially reversing the ischemic damage in an organ; or the methods of screening for modalities capable of improving or enhancing organ function, are comprising one or more steps of:
coupling or connecting the organ to an ex vivo or ex situ (machine) perfusion device;
adding to the perfusion solution a test modality, wherein the test modality is designed to reverse or to partially reverse a defect of or in an organ; or wherein the test modality is designed to reverse or to partially reverse ischemic damage in an organ; or wherein the test modality is designed to improve or enhance organ function;
monitoring the presence, level or concentration of 1 or more analytes in the perfusate, wherein the analytes are arginine, glutamate, glutamine, glucose, lactate, alanine, leucine and/or isoleucine.

The monitoring of the presence, level or concentration of the 1 or more analytes may include:
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine.

Alternatively, such methods of screening for modalities capable of reversing or partially reversing defects of an organ; or the methods of/for screening for modalities capable of reversing or partially reversing the ischemic damage in an organ; or the methods of screening for modalities capable of improving or enhancing organ function, are comprising one or more steps of:
coupling or connecting the organ to an ex vivo or ex situ (machine) perfusion device;
adding to the perfusion solution a test modality, wherein the test modality is designed to reverse or to partially reverse a defect of or in an organ; or wherein the test modality is designed to reverse or to partially reverse ischemic damage in an organ; or wherein the test modality is designed to improve or enhance organ function;
sampling the perfusate during or after initiating the (ex vivo or ex situ/machine) perfusion;
determining, measuring, assessing or analyzing the presence, level or concentration of 1 or more analytes in the perfusate sample(s), wherein the analytes are arginine, glutamate, glutamine, glucose, lactate, alanine, leucine and/or isoleucine; or wherein the analyte is arginine; or wherein the analytes are arginine and 1 or more of, glutamate, glutamine, glucose, lactate, alanine, leucine and/or isoleucine;
monitoring the viability or function of the perfused organ, wherein the perfused organ is determined to be viable, to remain viable, or to function properly when the determined, measured, assessed or analyzed presence, level or concentration of the 1 or more analytes is not or not significantly deviating from the control or reference level or concentration of the 1 or more analytes (such as representative for a healthy (ex vivo or ex situ/machine) perfused organ); or, alternatively, wherein the viability or function of the perfused organ is determined to be impacted when the determined, measured, assessed or analyzed presence, level or concentration of the 1 or more analytes is deviating or is significantly deviating from the control or reference level or concentration of the 1 or more analytes (such as representative for a healthy (ex vivo or ex situ/machine) perfused organ);

An additional step could be comprised in these methods:
selecting a test modality which is not or not significantly affecting or impacting the viability or function of the perfused organ.

In a further embodiment to any of the above methods referring to sampling the perfusate, the sampling of the perfusate is a single sampling at one time point or at at least one time point during or after start of the (ex vivo or ex situ/machine) perfusion, the sampling is at different single time points during or after start of the (ex vivo or ex situ/machine) perfusion, or the sampling is continuous during or after start of the (ex vivo or ex situ/machine) perfusion. “Sampling”, “obtaining a sample”, “taking a sample”, and “a sample obtained from” can be used interchangeably. A “sample”, “aliquot”, “specimen”, and “small amount or quantity” can be used interchangeably. Obviously, sampling the perfusate of an ex vivo or ex situ (machine) perfused organ implies that the organ has been coupled or connected to a (machine) perfusion device.

In a further embodiment to any of the above methods the organ is perfused under the same or similar conditions as the healthy (ex vivo or ex situ/machine) perfused organ. Herein, similar conditions are considered to include near identical conditions. Differences include e.g. slightly different concentration of one or more of the components of the perfusion solution, or slightly different perfusion conditions (e.g. slightly different temperature but overall within e.g. the definition of either “cold or hypothermic perfusion” or “warm or normothermic perfusion”). As such, similar conditions in this context are not expected to significantly change the viability or function of the perfused organ. Larger differences in the perfusion conditions may also be included. For instance, a red blood cell-based perfusion solution and whole blood-based perfusion solution where used in the work leading to the current invention; nevertheless these larger differences in perfusion conditions did not significantly change the viability or function of the perfused organ.

With “measuring, determining, assessing, analyzing, detecting, quantifying, sensing, etc.” (used interchangeably herein) the presence, level or concentration of an analyte of interest in a sample (such as in the perfusate or perfusate sample) is meant herein any analytical methodology capable of detecting the presence of the analyte of interest, and/or capable of detecting the relative quantity (level or concentration) of the analyte of interest in the sample.

With “monitoring” the presence, level or concentration of an analyte of interest in a sample is meant that the presence, level or concentration of the analyte of interest is followed over time, such as in samples taken at different time points, or taken continuously, during a process. During a process (such as ex vivo or ex situ (machine) perfusion of an organ), the presence, level or concentration of an analyte of interest may thus remain practically unchanged, may increase or may decrease (relative to the starting conditions, or relative to a reference or control level or concentration of an analyte of interest as determined specific for the process).

When in a process the presence, level or concentration of an analyte of interest is deviating from a reference or control level or concentration of the analyte of interest (e.g. as determined at one or more time points, during monitoring), this is indicative of the process deviating from the expected or desired process. Thus, when the presence, level or concentration of an analyte of interest is deviating from a reference or control level or concentration of the analyte of interest during (ex vivo or ex situ/machine) perfusion of a healthy or non-damaged organ, this is indicative of the occurrence of damage in the perfused organ, or is indicative of damage that has already occurred in the perfused organ (such as prior to start of the (ex vivo or ex situ/machine) perfusion). In particular, (significant) deviation of the presence, level or concentration of 1 or more of the analyte(s) of interest of the current invention relative to a reference or control level or concentration of the 1 or more analyte(s) of interest is indicative of (significant) ischemic damage occurring or having occurred in the perfused organ. Logically, the extent of deviation is linked to the extent of damage. As such, relatively minor deviations (1 to 5%, 1 to 10%, 1 to 20%, 1 to 25%, 1 to 30%) may still be considered as acceptable, i.e., perfused organs displaying such deviations may still be considered as sufficiently fit, sufficiently functional or of sufficient quality. When the deviations are significant, these will be considered as non-acceptable, i.e. perfused organs displaying such significant deviations will be considered as not sufficiently fit, not sufficiently or sub-optimally functional or of poor or bad quality, i.e. the viability or function of the perfused organ is impacted or significantly impacted. The limit and boundaries of acceptable deviations/significant deviations may furthermore shift with increasing clinical experience and understanding.

In the above description of the methods, the presence, level or concentration of the 1 or more analytes is compared to the control or reference level or concentration of the 1 or more analytes. The control or reference level or concentration of the 1 or more analytes can in one embodiment be representative for a healthy ex vivo or ex situ (machine) perfused organ. This obviously means that this comparison is performed per individual analyte. As will be described further hereinafter, other ways of establishing control or reference values of analytes are feasible. As such, each of these alternative control or reference analytes can be included in the above described methods in further defining the reference or control values.

The “presence, level or concentration of 1 or more analytes” in several individual embodiments is referred to as the presence, levels or concentrations of 1, 2, 3, 4, 5, 6, 7 or 8 analytes that are analyzed in a perfusate or perfusate sample. In particular, the presence, levels or concentrations of 1, 2, 3, 4, 5, 6, 7 or 8 of the analytes arginine, glutamate, glutamine, glucose, lactate, alanine, leucine and isoleucine is analyzed. In a particular case, the presence, level or concentration of 1 analyte is determined wherein the analyte is arginine.

Furthermore, such 2 analytes include, without necessarily being exhaustive:
arginine and glutamate; arginine and glutamine; arginine and glucose; arginine and lactate; arginine and alanine; glutamate and glutamine; glutamate and glucose; glutamate and lactate; glutamate and alanine; glutamine and glucose; glutamine and lactate; glutamine and alanine; glucose and lactate; glucose and alanine; lactate and alanine; arginine and leucine; glutamate and leucine; glutamine and leucine; glucose and leucine; lactate and leucine; alanine and leucine; arginine and isoleucine; glutamate and isoleucine; glutamine and isoleucine; glucose and isoleucine; lactate and isoleucine; alanine and isoleucine; and leucine and isoleucine.

Such 3 analytes include, without necessarily being exhaustive:
arginine, glutamate and glutamine; arginine, glutamate and glucose; arginine, glutamate and lactate; arginine, glutamate and alanine; arginine, glutamine and glucose; arginine, glutamine and lactate; arginine, glutamine and alanine; arginine, glucose and lactate; arginine, glucose and alanine; arginine, lactate and alanine; glutamate, glutamine and glucose; glutamate, glutamine and lactate; glutamate, glutamine and alanine; glutamate, glucose and lactate; glutamate, glucose and alanine; glutamate, lactate and alanine; glutamine, glucose and lactate; glutamine, glucose and alanine; glutamine, lactate and alanine; glucose, lactate and alanine; arginine, glutamate and leucine; arginine, glutamine and leucine; arginine, glucose and leucine; arginine, lactate and leucine; arginine, alanine and leucine; arginine, leucine and isoleucine; glutamate, glutamine and leucine; glutamate, glucose and leucine; glutamate, lactate and leucine; glutamate, alanine and leucine; glutamate, leucine and isoleucine; glutamine, glucose and leucine; glutamine, lactate and leucine; glutamine, alanine and leucine; glutamine, leucine and isoleucine; glucose, lactate and leucine; glucose, alanine and leucine; glucose, leucine and isoleucine; lactate, alanine and leucine; lactate, leucine and isoleucine; arginine, glutamate and isoleucine; arginine, glutamine and isoleucine; arginine, glucose and isoleucine; arginine, lactate and isoleucine; arginine, alanine and isoleucine; glutamate, glutamine and isoleucine; glutamate, glucose and isoleucine; glutamate, lactate and isoleucine; glutamate, alanine and isoleucine; glutamine, glucose and isoleucine; glutamine, lactate and isoleucine; glutamine, alanine and isoleucine; glucose, lactate and isoleucine; glucose, alanine and isoleucine; and lactate, alanine and isoleucine.

Such 4 analytes include, without necessarily being exhaustive:
arginine, glutamate, glutamine and glucose; arginine, glutamate, glutamine and lactate; arginine, glutamate, glutamine and alanine; arginine, glutamate, glucose and lactate; arginine, glutamate, glucose and alanine; arginine, glutamate, lactate and alanine; arginine, glutamine, glucose and lactate; arginine, glutamine, glucose and alanine; arginine, glutamine, lactate and alanine; arginine, glucose, lactate and alanine; glutamate, glutamine, glucose and lactate; glutamate, glutamine, glucose and alanine; glutamate, glutamine, lactate and alanine; glutamate, glucose, lactate and alanine; glutamine, glucose, lactate and alanine; arginine, glutamate, glutamine and leucine; arginine, glutamate, glucose and leucine; arginine, glutamate, lactate and leucine; arginine, glutamate, alanine and leucine; arginine, glutamine, glucose and leucine; arginine, glutamine, lactate and leucine; arginine, glutamine, alanine and leucine; arginine, glucose, lactate and leucine; arginine, glucose, alanine and leucine; arginine, lactate, alanine and leucine; arginine, glutamate, leucine and isoleucine; arginine, glutamine, leucine and isoleucine; arginine, glucose, leucine and isoleucine; arginine, lactate, leucine and isoleucine; arginine, alanine, leucine and isoleucine; glutamate, glutamine, glucose and leucine; glutamate, glutamine, lactate and leucine; glutamate, glutamine, alanine and leucine; glutamate, glucose, lactate and leucine; glutamate, glucose, alanine and leucine; glutamate, lactate, alanine and leucine; glutamate, glutamine, leucine and isoleucine; glutamate, glucose, leucine and isoleucine; glutamate, lactate, leucine and isoleucine; glutamate, alanine, leucine and isoleucine; glutamine, glucose, lactate and leucine; glutamine, glucose, alanine and leucine; glutamine, lactate, alanine and leucine; glutamine, glucose, leucine and isoleucine; glutamine, lactate, leucine and isoleucine; glutamine, alanine, leucine and isoleucine; glucose, lactate, alanine and leucine; glucose, lactate, leucine and isoleucine; glucose, alanine, leucine and isoleucine; lactate, alanine, leucine and isoleucine; arginine, glutamate, glutamine and isoleucine; arginine, glutamate, glucose and isoleucine; arginine, glutamate, lactate and isoleucine; arginine, glutamate, alanine and isoleucine; arginine, glutamine, glucose and isoleucine; arginine, glutamine, lactate and isoleucine; arginine, glutamine, alanine and isoleucine; arginine, glucose, lactate and isoleucine; arginine, glucose, alanine and isoleucine; arginine, lactate, alanine and isoleucine; glutamate, glutamine, glucose and isoleucine; glutamate, glutamine, lactate and isoleucine; glutamate, glutamine, alanine and isoleucine; glutamate, glucose, lactate and isoleucine; glutamate, glucose, alanine and isoleucine; glutamate, lactate, alanine and isoleucine; glutamine, glucose, lactate and isoleucine; glutamine, glucose, alanine and isoleucine; glutamine, lactate, alanine and isoleucine; and glucose, lactate, alanine and isoleucine.

Such 5 analytes include, without necessarily being exhaustive:
arginine, glutamate, glutamine, glucose and lactate; arginine, glutamate, glutamine, glucose and alanine; arginine, glutamate, glucose, lactate and alanine; arginine, glutamine, glucose, lactate and alanine; arginine, glutamate, glutamine, lactate and alanine; glutamate, glutamine, glucose, lactate and alanine; arginine, glutamate, glutamine, glucose and leucine; arginine, glutamate, glutamine, lactate and leucine; arginine, glutamate, glutamine, alanine and leucine; arginine, glutamine, glucose, lactate and leucine; arginine, glutamine, glucose, alanine and leucine; arginine, glutamine, lactate, alanine and leucine; arginine, glutamate, glutamine, leucine and isoleucine; arginine, glutamate, glucose, leucine and isoleucine; arginine, glutamate, lactate, leucine and isoleucine; arginine, glutamate, alanine, leucine and isoleucine; arginine, glutamine, glucose, leucine and isoleucine; arginine, glutamine, lactate, leucine and isoleucine; arginine, glutamine, alanine, leucine and isoleucine; arginine, glutamine, lactate, leucine and isoleucine; arginine, glutamine, alanine, leucine and isoleucine; arginine, lactate, alanine, leucine and isoleucine; glutamate, glutamine, glucose, lactate and leucine; glutamate, glutamine, glucose, alanine and leucine; glutamate, glucose, lactate, alanine and leucine; glutamate, glutamine, glucose, leucine and isoleucine; glutamate, glutamine, lactate, leucine and isoleucine; glutamate, glutamine, alanine, leucine and isoleucine; glutamate, glucose, lactate, leucine and isoleucine; glutamate, glucose, alanine, leucine and isoleucine; glutamate, lactate, alanine, leucine and isoleucine; glutamine, glucose, lactate, alanine and leucine; glutamine, glucose, lactate and leucine; glutamine, glucose, alanine and leucine; glutamine, lactate, alanine, leucine and isoleucine; glucose, lactate, alanine, leucine and isoleucine; arginine, glutamate, glutamine, glucose and isoleucine; arginine, glutamate, glutamine, lactate and isoleucine; arginine, glutamate, glutamine, alanine and isoleucine; arginine, glutamine, glucose, lactate and isoleucine; arginine, glutamine, glucose, alanine and isoleucine; arginine, glutamine, lactate, alanine and isoleucine; glutamate, glutamine, glucose, lactate and isoleucine; glutamate, glutamine, glucose, alanine and isoleucine; glutamate, glucose, lactate, alanine and isoleucine; glutamine, glucose, lactate, alanine and isoleucine; glutamine, glucose, lactate and isoleucine; and glutamine, glucose, alanine and isoleucine.

Such 6 analytes include, without necessarily being exhaustive: arginine, glutamate, glutamine, glucose, lactate and alanine; arginine, glutamate, glutamine, glucose, lactate and leucine; arginine, glutamate, glutamine, glucose, alanine and leucine; arginine, glutamine, glucose, lactate, alanine and leucine; arginine, glutamate, glucose, lactate, alanine and leucine; arginine, glutamate, glutamine, lactate, alanine and leucine; arginine, glutamate, glutamine, glucose, alanine and leucine; arginine, glutamate, glutamine, glucose, leucine and isoleucine; arginine, glutamate, glutamine, lactate, leucine and isoleucine; arginine, glutamate, glutamine, alanine, leucine and isoleucine; arginine, glutamine, glucose, lactate, leucine and isoleucine; arginine, glutamine, glucose, alanine, leucine and isoleucine; arginine, glucose, lactate, alanine, leucine and isoleucine; glutamate, glutamine, glucose, lactate, alanine and leucine; glutamate, glutamine, glucose, lactate, leucine and isoleucine; glutamate, glutamine, glucose, alanine, leucine and isoleucine; glutamate, glucose, lactate, alanine, leucine and isoleucine; glutamate, glutamine, lactate, alanine, leucine and isoleucine; glutamate, glutamine, glucose, lactate, leucine and isoleucine; glutamate, glutamine, glucose, alanine, leucine and isoleucine; glutamine, glucose, lactate, alanine, leucine and isoleucine; arginine, glutamate, glutamine, glucose, lactate and isoleucine; arginine, glutamate, glutamine, glucose, alanine and isoleucine; arginine, glutamine, glucose, lactate, alanine and isoleucine; arginine, glutamate, glucose, lactate, alanine and isoleucine; arginine, glutamate, glutamine, lactate, alanine and isoleucine; arginine, glutamate, glutamine, glucose, alanine and isoleucine; and glutamate, glutamine, glucose, lactate, alanine and isoleucine.

Such 7 analytes include, without necessarily being exhaustive: arginine, glutamate, glutamine, glucose, lactate, alanine and leucine; arginine, glutamate, glutamine, glucose, lactate, alanine and isoleucine; arginine, glutamine, glucose, lactate, alanine, leucine and isoleucine; arginine, glutamate, glucose, lactate, alanine, leucine and isoleucine; arginine, glutamate, glutamine, lactate, alanine, leucine and isoleucine; arginine, glutamate, glutamine, glucose, lactate, leucine and isoleucine; arginine, glutamate, glutamine, glucose, alanine, leucine and isoleucine; and glutamate, glutamine, glucose, lactate, alanine, leucine and isoleucine.

Such 8 analytes include: arginine, glutamate, glutamine, glucose, lactate, alanine, leucine and isoleucine.

In one further embodiment, the presence, levels or concentrations of arginine and of one or more of glutamate, glutamine, glucose, lactate, alanine, leucine and isoleucine are analyzed in the perfusate or perfusate sample(s). Such combinations of analytes to be analyzed include, without necessarily being exhaustive, all above listed combinations of 2 to 8 analytes comprising arginine.

In one further embodiment, the presence, levels or concentrations of glutamate and of one or more of arginine, glutamine, glucose, lactate, alanine, leucine and isoleucine are analyzed in the perfusate or perfusate sample(s). Such combinations of analytes to be analyzed include, without necessarily being exhaustive, all above listed combinations of 2 to 8 analytes comprising glutamate.

In one further embodiment, the presence, levels or concentrations of glutamine and of one or more of arginine, glutamate, glucose, lactate, alanine, leucine and isoleucine are analyzed in the perfusate or perfusate sample(s). Such combinations of analytes to be analyzed include, without necessarily being exhaustive, all above listed combinations of 2 to 8 analytes comprising glutamine.

In one further embodiment, the presence, levels or concentrations of glucose and of one or more of arginine, glutamate, glutamine, lactate, alanine, leucine and isoleucine are analyzed in the perfusate or perfusate sample(s). Such combinations of analytes to be analyzed include, without necessarily being exhaustive, all above listed combinations of 2 to 8 analytes comprising glucose.

In one further embodiment, the presence, levels or concentrations of lactate and of one or more of arginine, glutamate, glutamine, glucose, alanine, leucine and isoleucine are analyzed in the perfusate or perfusate sample(s). Such combinations of analytes to be analyzed include, without necessarily being exhaustive, all above listed combinations of 2 to 8 analytes comprising lactate.

In one further embodiment, the presence, levels or concentrations of alanine and of one or more of arginine, glutamate, glutamine, glucose, lactate, leucine and isoleucine are analyzed in the perfusate or perfusate sample(s). Such combinations of analytes to be analyzed include, without necessarily being exhaustive, all above listed combinations of 2 to 8 analytes comprising alanine.

In one further embodiment, the presence, levels or concentrations of leucine and of one or more of arginine, glutamate, glutamine, glucose, lactate, alanine, and isoleucine are analyzed in the perfusate or perfusate sample(s). Such combinations of analytes to be analyzed include, without necessarily being exhaustive, all above listed combinations of 2 to 8 analytes comprising leucine.

In one further embodiment, the presence, levels or concentrations of isoleucine and of one or more of arginine, glutamate, glutamine, glucose, lactate, alanine, and leucine are analyzed in the perfusate or perfusate sample(s). Such combinations of analytes to be analyzed include, without necessarily being exhaustive, all above listed combinations of 2 to 8 analytes comprising isoleucine.

In a further embodiment to any method, aspect or other embodiment herein, the presence, levels or concentrations of glucose is analyzed in the perfusate or perfusate sample(s), and of one or more of arginine, glutamate, glutamine, alanine and isoleucine are analyzed in the perfusate or perfusate sample(s). Such combinations of analytes to be analyzed include, without necessarily being exhaustive, all above listed combinations comprising glucose and 1 or more of arginine, glutamate, glutamine, alanine and isoleucine, but excluding lactate and/or leucine.

In a further embodiment to any method, aspect or other embodiment herein, the presence, levels or concentrations of lactate, and of one or more of arginine, glutamate, glutamine, alanine, leucine and isoleucine are analyzed in the perfusate or perfusate sample(s). Such combinations of analytes to be analyzed include, without necessarily being exhaustive, all above listed combinations comprising lactate one or more of arginine, glutamate, glutamine, alanine, leucine and isoleucine, but excluding glucose.

In a further embodiment to any method, aspect or other embodiment herein, the presence, levels or concentrations of leucine is analyzed in the perfusate or perfusate sample(s), and of one or more of arginine, glutamate, glutamine, alanine, lactate and isoleucine are analyzed in the perfusate or perfusate sample(s). Such combinations of analytes to be analyzed include, without necessarily being exhaustive, all above listed combinations comprising leucine one or more of arginine, glutamate, glutamine, alanine, lactate and isoleucine, but excluding glucose.

In a further embodiment to any method, aspect or other embodiment herein, the presence, levels or concentrations of glucose and lactate are analyzed in the perfusate or perfusate sample(s), and of one or more of arginine, glutamate, glutamine, alanine, leucine, and isoleucine are analyzed in the perfusate or perfusate sample(s). Such combinations of analytes to be analyzed include, without necessarily being exhaustive, all above listed combinations comprising glucose and lactate, and one or more of arginine, glutamate, glutamine, alanine, leucine, and isoleucine.

In a further embodiment to any method, aspect or other embodiment herein, the presence, levels or concentrations of glucose and leucine are analyzed in the perfusate or perfusate sample(s), and of one or more of arginine, glutamate, glutamine, lactate, alanine, and isoleucine are analyzed in the perfusate or perfusate sample(s). Such combinations of analytes to be analyzed include, without necessarily being exhaustive, all above listed combinations comprising glucose and leucine, and one or more of arginine, glutamate, glutamine, lactate, alanine, and isoleucine.

In a series of further embodiments to any method, aspect or other embodiment herein, the changes in presence, level or concentration of individual analytes during ex vivo or ex situ machine perfusion of a kidney is described in more detail hereinafter.

The presence, levels or concentrations of arginine in the perfusate of a healthy or non-ischemic perfused kidney are (relatively) stable. The presence, levels or concentrations of arginine in the perfusate of a damaged or ischemic (both cold or warm ischemic) kidney are (significantly) decreasing early during the perfusion (setting in at 60 min or earlier than 60 min after start of the perfusion) and then remain relatively constant; under the herein applied perfusion conditions the arginine is in fact fully consumed by ischemic kidneys.

The presence, levels or concentrations of glucose in the perfusate of a healthy or non-ischemic perfused kidney are decreasing, more in particular the decrease is setting in between 120 and 180 min (including at 120 min, at 180 min) after start of the perfusion. The presence, levels or concentrations of glucose in the perfusate of a cold ischemic kidney are decreasing with kinetics similar as to a healthy kidney, but with a more extensive decrease compared to a healthy kidney. The presence, levels or concentrations of glucose in the perfusate of a warm ischemic kidney are (significantly) decreasing early during the perfusion (setting in at 60 min or earlier than 60 min after start of the perfusion) and then remain relatively constant.

The presence, levels or concentrations of glutamate in the perfusate of a healthy or non-ischemic kidney are (significantly) decreasing, more in particular the decrease is setting in between 60 and 120 min (including at 60 min, at 120 min) after start of the perfusion, and then remain relatively constant. The presence, levels or concentrations of glutamate in the perfusate of a cold ischemic kidney are decreasing with kinetics similar as to a healthy kidney, but with a less extensive decrease compared to a healthy kidney. In contrast, the presence, levels or concentrations of glutamate in the perfusate of a warm ischemic kidney are (significantly) increasing early during the perfusion (setting in between 60 and 120 min, including at 60 min, at 120 min, after start of the perfusion) and then remain relatively constant.

The presence, levels or concentrations of glutamine in the perfusate of a healthy or non-ischemic perfused kidney are (significantly) decreasing (setting in between 60 and 120 min, including at 60 min, at 120 min, after start of the perfusion). The decrease in presence, levels or concentrations of glutamine are less significant for cold ischemic kidneys compared to healthy kidneys and the difference is setting in between 120 and 180 min (including at 120 min, at 180 min) after start of the perfusion. The decrease in presence, levels or concentrations of glutamine are less significant for warm ischemic kidneys compared to cold ischemic kidneys and healthy kidneys and the difference is setting from 120 min after start of the perfusion.

The presence, levels or concentrations of lactate in the perfusate of kidneys are (significantly) increasing for healthy or non-ischemic kidneys. The increase in presence, levels or concentrations of lactate is less significant in the perfusate of warm ischemic kidneys compared to healthy kidneys and the difference is setting from 180 min after start of the perfusion. The increase in presence, levels or concentrations of lactate is less significant in the perfusate of cold ischemic kidneys compared to warm ischemic kidneys and healthy kidneys, and the difference is setting from 120 min after start of the perfusion.

The presence, levels or concentrations of alanine in the perfusate of kidneys are (significantly) increasing for healthy or non-ischemic kidneys, for cold ischemic kidneys and for warm ischemic kidneys. The increase is more pronounced for warm ischemic kidneys compared to healthy kidneys and the difference is setting in early during perfusion (setting in at 60 min or earlier than 60 min after start of the perfusion).

The increase is less pronounced for cold ischemic kidneys compared to healthy kidneys and the difference is setting in between 60 and 120 min (including at 60 min, at 120 min) after start of the perfusion.

The presence, levels or concentrations of leucine in the perfusate of kidneys are (significantly) increasing in all kidney, setting in at 60 min or earlier than 60 min after start of the perfusion. The increase is, however, more significant for healthy or non-ischemic kidneys compared to both warm and cold ischemic kidneys.

The presence, levels or concentrations of isoleucine in the perfusate of kidneys are (significantly) increasing in all kidney, setting in at 60 min or earlier than 60 min after start of the perfusion. The increase is, however, more significant for healthy or non-ischemic kidneys compared to both warm and cold ischemic kidneys.

In a further embodiment to any aspect or other embodiment herein, the presence, levels or concentrations of individual analytes during ex vivo or ex situ machine perfusion of a kidney is determined or analyzed at 120 min, or at a time point between 90 and 150 min, or at a time point between 100 and 140 min, or at time point between 105 and 135 min, after start of the perfusion. These presence, levels or concentrations can subsequently be compared to a relevant reference value as described herein.

Arginine, Arg, arg, or R; glucose, Glc or glc; glutamate, glutamic acid, Glu, glu or E; glutamine, Gln, gln or Q; lactate, lactic acid, Lac or lac; alanine, Ala, ala, or A; leucine, Leu, leu or L; and isoleucine, Ile, ile or I, are each per group used interchangeably.

### Organ Preservation, Organ Perfusion, Hypothermic Perfusion, Sub-Normothermic Perfusion, Normothermic Perfusion, Perfusion Solutions, Perfusate and Perfusate Sample, Donor, Transplantation

During/after organ procurement, an organ preservation solution or organ flushing solution is initially perfused in the organ, or the organ is flushed with this solution through its artery(ies) and or vein(s), usually, but not necessarily, under hypothermic conditions; the flushing can be performed in situ or ex situ; the organ is then usually stored under hypothermia during static storage.

Organ perfusion solutions in particular are circulated over the organ, such as circulated in an ex vivo or ex situ perfusion or machine perfusion setting. Organ perfusion can be under hypothermic (ca. 0-10° C.) or normothermic conditions (ca. 18-20-35-37° C.); a (stepwise) switch from hypothermic over sub-normothermic to normothermic conditions is likewise possible. Organ perfusion solutions have as aim, as do organ preservation solutions, to preserve the organ in an as good condition as possible.

For purposes of cold storage of an organ, a preservation solution is infused into the organ followed by storage of the infused organ under cold/hypothermic conditions. Under hypothermia, the metabolic activity of an organ is reduced. Oxygen consumption in e.g. a kidney under hypothermic perfusion is diminished to about 5-10% of that at body temperature. Under cold perfusion conditions, the perfusion solution does therefore not need to be near-physiological. Under sub-normothermic, or warm or normothermic perfusion conditions, metabolism of the organ is occurring at a near normal rate and sufficient metabolic support components have to be included in the perfusion solution circulated over the perfused organ. Sub-normothermic and normothermic perfusion solutions therefore provide a more physiological-like environment to the perfused organ, including oxygenation by blood/erythrocyte-based oxygenation or via an oxygen-carrier (an oxygen carrier in generally is included when the perfusion temperature is 20 to 25° C. or higher). If not attended to timely, pump failure under normothermic perfusion may lead to organ loss; a risk that is much lower under hypothermic perfusion.

An organ may be subjected both to ex vivo hypothermia (statically or under perfusion; such as e.g. during organ transport and/or organ storage after harvesting or procurement) and ex vivo normothermia (under perfusion). Initial observations of possible rescue of ex-vivo hypothermically perfused kidneys discarded for transplantation by subsequent normothermic perfusion led Kabagambe et al. 2019 (Transplantation 103: 392-400) to the idea of setting up a clinical trial to further validate the initial observations. The switch from hypothermia to normothermia may be gradually imposed, and appears possible with perfusion solutions not comprising blood components, erythrocytes, or artificial oxygen carriers, as demonstrated for kidneys using an oxygenated solution based on the Steen solution as perfusion solution (Minor et al. 2020, Am J Transplant 20:1192-1195).

Organ perfusion solutions can be synthetic, acellular, can comprise e.g. red blood cells (or erythrocytes or RBCs) or whole blood (WB), or can comprise artificial hemoglobin (e.g. Bodewes et al. 2021, Int J Mol Sci 22:235). Organ perfusion solutions may be adaptations of organ flushing or organ preservation solutions. Organ perfusion solutions or organ preservation solutions may initially be or have been developed for one target organ but later on find or have found applicability in a wider set of target organs.

The most widely utilized preservation solution (in particular cold storage preservation solution) perhaps is the University of Wisconsin (UW) solution, which is applied in preservation of heart, liver, kidney, pancreas, small bowel, etc. The UW solution, with or without minor adaptations, is also been applied as organ perfusion solution. The UW solution has an osmolality of 320 mOsm and pH 7.4 at room temperature and is composed of the following (concentration of some components may vary among manufacturers): potassium ˜120-135 mmol/L, sodium ˜30-35 mmol/L, magnesium 5 mmol/L, lactobionate (as lactone) 100 mmol/L, phosphate 25 mmol/L, sulphate 5 mmol/L, raffinose 30 mmol/L, adenosine 5 mmol/L, allopurinol 1 mmol/L, glutathione 3 mmol/L, insulin 100 U/L, dexamethasone 8 mg/L, hydroxyethyl starch (HES) (pentafraction) 50 g/L. Prior to use, components such as penicillin G, regular insulin, dexamethasone and/or bactrim can be (aseptically) added. Variants of the UW solution include RPS-96 (lacking HES), dextran 40 UW (in which HES is replaced by dextran 40), perfluorocarbons (PFC) UW, hyperbranched polyglycerol (HPG) UW (solution (in which HES is replaced by HPG), and sodium lactobionate sucrose (SLS) UW (in which raffinose is replaced by sucrose) (summarized in Chen et al. 2019, Cell Transplantation 28:1472-1489). The components of UW solution are utilized to prevent cellular edema, cell destruction, maintain organ metabolic potential, and to maximize organ function after perfusion is re-established.

Other perfusion or preservation solutions include Euro-Collins (or EC), Celsior, Custodiol (or HTK: histidine tryptophane ketoglutarate), IGL-1 (Institute Georges Lopez-1), and hypertonic citrate adenine (HC-A) (compositions described in Table 1 of Voigt et al. 2013, Progress in Transplantation 23:383-391; and/or in Table 1 of Chen et al. 2019, Cell Transplantation 28:1472-1489). Lee and Mangino 2009 (Organogenesis 5:105-112) in addition describe in Table 1 the composition of Belzer's MPS solution and Polysol solution. The compositions of the ET-Kyoto (extracellular-type-Kyoto) solution and Perfadex solution are shown in Table 1 of Okamoto et al. 2011 (Transplantation Proceedings 43:1525-1528). The extracellular-like Krebs-Henseleit (KH) solution composition is given in Table 1 of van der Heijden et al. 1999 (Clinical Science 97:45-57). Yet further preservation solutions include phosphate buffered sucrose (PBS), 140, HP16, HBS, B2, Lifor, Ecosol, Biolasol, renal preservation solution 2 (RPS-2), F-M, AQIXRS-1, WMO-II, CZ-1, and SCOT solution (Solution de Conservation des Organes et des Tissus) (see Chen et al. 2019, Cell Transplantation 28:1472-1489 for more details). The use of IGL-1 in kidney perfusion, and comparison with UW and/or HTK is extensively reviewed by De Beule et al. 2021 (Am J Transplant 21:830-837) and Habran et al. 2020 (PLoS One 15:e0231019).

Further additives to these solutions that have been employed include, amongst other, endothelial receptor antagonist, rho-kinase inhibitor HA1077, melagatran, trophic factors (such as BNP-1, SP, NGF-b, IGF-1, EGF, HGF, IGF-1), recombinant human BMP-7, TNF-receptor fusion protein, caspase-3 siRNA, MMP-2 siRNA, ICAM-1 antisense oligodeoxynucleotide, taurine, ranolazine, verapamil, trimetazidine, trolox, deferoxamine, ascorbate, lecithinized superoxide dismutase, edaravone, mitoQ, quinacrine, nitroprusside, phosphoramidon, N-acetylcysteine, selenium, nicaraven, propofol, prostaglandin E1, and tanshinone IIA (see e.g. Table 2 of Chen et al. 2019, Cell Transplantation 28:1472-1489). The addition of oxygen during hypothermic MP has been reviewed by Darius et al. 2021 (Biomedicines 9:993).

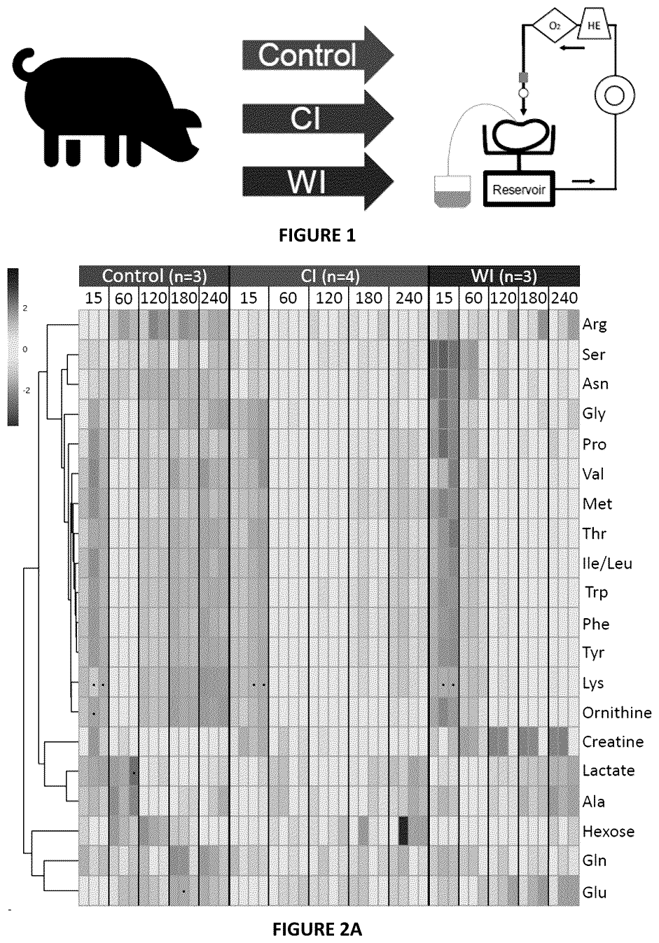

The nowadays most widely used solutions in hypothermic kidney preservation are EC, UW, HTK, Celsior, HC-A and IGL-1, as these appear to perform with comparable post-transplant outcomes. For normothermic perfusion of kidneys, Pool et al. 2021 (PLoS ONE 16:e251595) compared 4 different perfusion solutions all containing autologous red blood cells. Elliott et al. 2021 (Am J Transplant 21:1382-1390) provides in their FIG. 1 and Table 1 an overview of normothermic machine perfusion solution components and their function. A standard perfusion protocol has currently not been reached.

Numerous MP systems nowadays exist and are either portable or non-portable; and either pressure-driven or flow-driven. Portable devices for hypothermic kidney MP include the LifePort Kidney Transporter (Organ Recovery Systems, Itasca, IL, USA), the Kidney Assist Transporter (Organ Assist BV, Groningen, The Netherlands) and the WAVES machine (Institut Georges Lopez, Lissieu, France). Non-portable devices for hypothermic MP include the RM3 and RM4 device (Waters Medical Systems, Rochester, MN, USA) and VitaSmart (Bridge to Life, Northbrook, IL, USA) (Darius et al. 2021, Biomedicines 9:993).

Elliott et al. 2021 (Am J Transplant 21:1382-1390) refer to some normothermic MP systems: Kidney Assisti” Device (Organ Assist, CE marked; prototype portable normothermic kidney machine perfusion system under development by OrganOx); Organ Assist is commercializing the XVIVO's Kidney Assist perfusion machine for hypo- to normothermic kidney perfusion.

Different classes of organ donors can be discerned: (i) donor after brain death (DBD): a deceased donor for whom death has been determined by neurological criteria; also referred to as deceased heart-beating donor; (ii) donor after circulatory death (DCD): a deceased organ donor for whom death has been determined by circulatory and respiratory criteria; also referred to as deceased non-heart-beating donor; (iii) living donor: a living human being from whom cells, tissue or organs have been removed for the purpose of transplantation.

With transplantation is meant the transfer, transplantation or engraftment of human cells, tissues or organs from a donor to a recipient with the aim of restoring function(s) in the recipient's body. When transplantation is performed between different species (for instance, animal to human), it is referred to as xenotransplantation.

### Perfusate Sampling and Sampling Method, Reference Values

The perfusion solution or perfusate circulated through a perfused organ can generally be divided in the perfusate entering the perfused organ, also termed herein the afferent perfusate; and the perfusate leaving the perfused organ, also termed herein the efferent perfusate. In general the (afferent) perfusate is entering the perfused organ via an artery (or portal vein), and can alternatively be termed arterial perfusate; and the (efferent) perfusate is leaving the perfused organ via a vein, and can alternatively be termed venous perfusate. As a consequence of biochemical reactions in and/or metabolic activity of the perfused organ, the composition of the afferent perfusate changes as it passes through the perfused organ as the latter takes up, consumes, or absorbs components from the perfusate on the one hand, and releases or secretes (other) components to the perfusate on the other hand. The composition of the perfusate thus gradually changes over time during perfusion. Such changes can be analyzed, determined, measured or assessed, and are indicative of the underlying metabolic activity (healthy profile, or profile modified by an underlying damage or injury) of the perfused organ. As such, the concentration of some components either remains unchanged during (repeated) passage through the perfused organ (no uptake by the perfused organ, or uptake and release by the perfused organ are in equilibrium); decreases during (repeated) passage through the perfused organ (uptake or net uptake of the component(s) by the perfused organ); or increases during (repeated) passage through the perfused organ (secretion or net secretion of the component(s) by the perfused organ). In view of the perfusion circuit being closed (except for urine deposition—although urine can be recirculated as well (Weissenbacher et al. 2021, Am J Transplant 21:1740-1753) and oxygenation, and glucose and glutamine supply to the perfusate in the herein applied perfusion conditions—see Example 1.6), the perfusate sample at a given time point can in particular be an afferent perfusate sample, an efferent perfusate sample, or both, or generally be referred to as the perfusate sample (as the difference in composition of the afferent and efferent perfusate at the same time point will be minor).

When initiating ex vivo or ex situ perfusion of an organ, the perfusion solution will replace (or flush) any fluids present in the organ in the perfusion trajectory and the organ will in generally “equilibrate” with the perfusion solution. It was observed herein (see Example 1.9) that this so-called wash-out fraction (of the perfused organ) causes changes to the initial composition of the perfusion solution (i.e. the composition of the perfusion solution prior to start of the ex vivo or ex situ perfusion, or prior to contact with the to-be perfused organ), thus potentially obscuring interpretation of initial changes in concentration of individual analytes of interest in the perfusate (changes truly due to the metabolic activity of the perfused organ). It was further observed that changes in concentration of individual analytes of interest in the perfusate could be reliably determined once the wash-out fraction of the perfused organ and the initial perfusion solution were sufficiently mixed or were homogenized by the perfusion process. This does, however, not exclude such initial changes of being informative.

In view of the above, a further aspect of the invention relates in general to methods of analyzing the perfusate of an organ (including but explicitly not limited to a kidney) perfused ex vivo or ex situ with a perfusion solution, such methods including or comprising:
sampling the perfusate or obtaining a sample from the perfusate at different time points during the ex vivo or ex situ (machine) perfusion; and
determining a reference time point which is the time point during the ex vivo or ex situ (machine) perfusion at which the organ wash-out fraction stops, ceases or halts changing the (initial) perfusate composition; or alternatively at which the organ wash-out fraction has homogenized with the (initial) perfusion solution.

Such methods can further optionally comprise:
determining, defining, or setting the presence, level or concentration of an analyte of interest as measured or determined in the perfusate at the reference time point as reference or control level or concentration of the analyte of interest in the perfusate.

Such methods may comprise further steps such as:
analyzing, measuring, determining, or assessing (the concentration of) an analyte of interest in the obtained perfusate samples; and/or
analyzing, measuring, determining, or assessing changes in concentration of an analyte of interest in the obtained perfusate samples relative to the concentration of the analyte of interest (as analyzed, measured, determined, or assessed) at the determined reference time point.

In an alternative aspect of the invention, such methods of analyzing the perfusate of an organ perfused ex vivo or ex situ with a perfusion solution, are including or comprising:
sampling the perfusate or obtaining a sample from the perfusate at different time points during the ex vivo or ex situ (machine) perfusion;
analyzing, measuring, determining, or assessing (the concentration of) an analyte of interest in the obtained perfusate samples; and
analyzing, measuring, determining, or assessing changes in concentration of an analyte of interest in the obtained perfusate samples relative to the concentration of the analyte of interest (as analyzed, measured, determined, or assessed) at the determined reference time point which is the time point during the ex vivo or ex situ machine perfusion at which the organ wash-out fraction stops, ceases or halts changing the (initial) perfusate composition; or alternatively at which the organ wash-out fraction has homogenized with the (initial) perfusion solution.

In the above method, the reference time point will depend on the organ size, or at least on the size of the wash-out fraction of the organ. In the case of pig kidneys, the reference time point is reached at about 10 to 20 minutes, or at about 15 minutes after start of the perfusion.

As an alternative, (changes in) the concentration of an analyte of interest can be compared to a pre-defined reference concentration. Such reference concentration can in one embodiment be determined as an average value of analyte of interest concentrations in samples obtained from a (sufficiently large, or representative) set of different conditions and time points during or after start of ex vivo or ex situ (machine) perfusion.

Alternatively, such reference concentration is in another embodiment determined as an average value of analyte of interest concentration at a given time point during ex vivo or ex situ (machine) perfusion under a given condition. Thus, herein, the analyte of interest concentration is determined in a (sufficiently large, or representative) set of samples obtained at a single time point during or after start of ex vivo or ex situ (machine) perfusion, and the average value of analyte of interest concentration at that time point is then calculated or determined. The analyte of interest concentration in a test sample determined at a given time point during or after start of ex vivo or ex situ (machine) perfusion can then be compared to the reference concentration of that same analyte determined for the same given time point during or after start of ex vivo or ex situ (machine) perfusion, and this with the reference and test ex vivo or ex situ (machine) perfusion conditions being the same or near identical.

Thus, in a further alternative aspect of the invention, such methods of analyzing the perfusate of an organ perfused ex vivo or ex situ with a perfusion solution, are including or comprising:
sampling the perfusate or obtaining a sample from the perfusate at different time points during the ex vivo or ex situ (machine) perfusion;
analyzing, measuring, determining, or assessing (the level or concentration of) an analyte of interest in the obtained perfusate samples; and
analyzing, measuring, determining, or assessing changes in level or concentration of an analyte of interest in the obtained perfusate samples relative to a pre-determined reference level or concentration of the analyte of interest.

The “different time points during the ex vivo or ex situ machine perfusion” may include a time point “zero”, i.e. just at the start of the ex vivo or ex situ machine perfusion.

The “analyte of interest” can be any molecule present (already present or becoming present) in the perfusate such as a metabolite, DNA or RNA (e.g. as secreted by the perfused organ during the perfusion), proteins, etc., and include the herein identified/defined one or more of arginine, glutamate, glutamine, glucose, lactate, alanine, leucine and/or isoleucine.

### Organ Diagnostic Apparatus or Device, Kit

In one aspect, the hereinabove described methods can be performed on or by an organ diagnostic apparatus or device. In particular, the organ diagnostic apparatus or device in one embodiment comprises a unit or module capable of detecting, measuring, quantifying or sensing (such as in-line detection, measurement, quantification or sensing) of the herein described metabolites in the perfusate (afferent and/or efferent perfusate). The organ diagnostic apparatus or device may further comprise a unit or module displaying and/or recording (such as by automatically logging or storing on any type of electronic or computer-readable memory device) and/or transmitting (such as by wireless transmission; such as to a receiving display and/or recording unit or module) the detected, measured, quantified or sensed amount of the herein described metabolites in the (afferent and/or efferent) perfusate. Said display and/or recording unit or module may be a separate unit or module of the organ diagnostic apparatus or device, or may be integrated in the unit or module capable of detecting, measuring, quantifying or sensing (such as in-line detection, measurement, quantification or sensing) of the herein described metabolites in the (afferent and/or efferent) perfusate. Said organ diagnostic apparatus or device may be a stand-alone apparatus or device and may optionally comprise one or more further (diagnostic) units, modules or sensors capable of sensing e.g. one or more of oxygen levels, carbon dioxide levels, temperature, pH, vascular resistance, levels of lactate, production of urine (in case of the organ being a kidney), etc.

In one embodiment, the organ diagnostic apparatus or device is integrated in/with or part of an (organ) (machine) perfusion device or apparatus which includes further components such as units, modules, sensors and/or controllers of the (organ) perfusion system—as such the organ diagnostic apparatus or device is not a stand-alone apparatus or device.

In another embodiment, the organ diagnostic apparatus or device is integrated in/with or part of an organ transporter which allows for transportation of an organ over long distances. An organ transporter may include features of an organ perfusion device or apparatus, such as sensors and temperature controllers, and/or organ cassette interface features. It may therefore also include a unit or module capable of detecting, measuring, quantifying or sensing (such as in-line detection, measurement, quantification or sensing) of the herein described metabolites in the perfusate (afferent and/or efferent perfusate). It may therefore optionally also include a unit or module displaying and/or recording (such as by automatically logging or storing on any type of electronic or computer-readable memory device) and/or transmitting (such as by wireless transmission; such as to a receiving display and/or recording unit or module) the detected, measured, quantified or sensed amount of the herein described metabolites in the (afferent and/or efferent) perfusate.

In another embodiment, the organ diagnostic apparatus or device is integrated in/with or part of an organ cassette which allows to easily and safely move an organ between apparatus for perfusing, storing, analyzing and/or transporting the organ. An organ cassette may be configured to provide uninterrupted sterile conditions and efficient heat transfer during transport, recovery, analysis and storage, including transition between e.g. an organ transporter, a machine perfusion apparatus and a stand-alone organ diagnostic apparatus.

The organ diagnostic apparatus or device, whether or not stand-alone, may be networked to permit remote management, tracking and monitoring of the location and therapeutic and diagnostic parameters (such as the herein described parameters, i.e. metabolites in the perfusate (afferent and/or efferent perfusate)) of the organ being stored or transported (e.g. involving wireless communications setup to provide real-time data). The information systems may be used to compile historical data of organ transport and storage, and provide cross-referencing with hospital and e.g. the United Network for Organ Sharing (UNOS), Eurotransplant, Scandiatransplant, South Alliance for Transplants (SAT), etc., data on the donor and recipient. The systems may also provide outcome data to allow for ready research of perfusion parameters and transplant outcomes.

The organ diagnostic apparatus or device, whether or not stand-alone, may provide an organ viability index based on the diagnosed parameters (such as the herein described parameters, i.e. metabolites in the perfusate (afferent and/or efferent perfusate)) and optionally based on including information, such as age, gender, blood type of the donor and any expanded criteria; organ information, such as organ collection date and time, warm ischemia time, cold ischemia time and vascular resistance; apparatus information, such as flow rate, elapsed time the pump has been operating and pressure; and other identifiers such as UNOS, Eurotransplant, Scandiatransplant, SAT, etc., number and physician(s) in charge.

The invention thus relates in a further aspect to organ diagnostic apparatuses or devices comprising a unit or module, in particular a diagnostic unit or module, for detecting, measuring, quantifying, sensing or assessing one or more analytes present in a perfusate sample of an organ ex situ perfused with a perfusion solution, wherein the one or more analytes are arginine, glutamate, glutamine, glucose, lactate, alanine, leucine and/or isoleucine, as described in detail hereinabove. In a further particular embodiment, such organ diagnostic apparatus or device may comprise a further diagnostic unit or module. In another particular embodiment, such organ diagnostic apparatus or device is a stand-alone apparatus or device, or is integrated in or with a machine perfusion apparatus or device, in or with an organ transporter, or in or with an organ cassette.

In a specific aspect, the invention relates to a machine perfusion apparatus or device, an organ transporter, or an organ cassette, further characterized in that it is comprising a unit or module, in particular a diagnostic unit or module, for detecting, measuring, quantifying, sensing or assessing the presence, level or concentration of 1 or more analytes present in the perfusate or in a perfusate sample of an organ ex situ perfused with a perfusion solution, wherein the one or more analytes are arginine, glutamate, glutamine, glucose, lactate, alanine, leucine and/or isoleucine, as described in detail hereinabove.

The invention relates in a further aspect to analytical kits for, for use in, or for use in a method of/for detecting, measuring, quantifying, sensing or assessing one or more analytes present in a perfusate sample of an organ ex situ perfused with a perfusion solution, wherein the one or more analytes are arginine, glutamate, glutamine, glucose, lactate, alanine, leucine and/or isoleucine, as described/defined in detail hereinabove. Such analytical kits typically comprise one or more reagents (e.g. required for the detection of an analyte) and/or one or more analytical tools (e.g. a resin for capturing an analyte).

Instructions on how to apply the analytical kit in practice are usually also part of the kit. Such analytical kits can comprise re-usable part(s) and/or can comprise single use or disposable part(s).

### EXAMPLES

### 1. Materials and Methods

### 1.1. Animals

Male prepubescent pigs (TOPIGS TN70, Tojapigs, the Netherlands), a crossbreed of Landrace and York, weighing 35-45 kg were used. Pigs were maintained in a specific-pathogen-free animal facility with ad libitum access to food and water and were acclimated for a minimum of 2 days before experiments. Pigs were fasted for 12 hours before experiments with ad libitum access to water. All experiments were performed according to the European guidelines (Directive 2010/63/EU of the European Parliament and of the Council of 22 Sep. 2010) and were approved by the Animal Ethics Committee (P209/2017, KU Leuven, Belgium).

### 1.2. Pig Experiments

Pigs were sedated by an intramuscular injection of Tiletamine/Zolazepam (8 mg/kg, Zoletil®, Virbac, Belgium) Xylazine (2 mg/kg, Xylazine®, VMD pharma, Belgium). They were anaesthetised by inhalation of isoflurane (1% Isovet®, Piramal Critical Care B.V., Belgium) followed by orotracheal intubation. Anaesthesia was maintained by isoflurane and continuous infusion of fentanyl (8 μg/kg, Fentanyl®, Janssen Pharmaceutica, Belgium). After a midline laparotomy, both kidneys were dissected free from the surrounding tissues keeping the renal vessels and ureter as long as possible. After procurement the renal artery was cannulated (14 French armoured cannula, Biomedicus, Medtronic, Belgium). The arterial cannula was secured with a purse string suture to allow connection to the isolated kidney perfusion set-up. The kidney was flushed with 250 mL of cold (˜2° C.) preservation solution (Institut George Lopez-1 solution (IGL-1), IGL, France) to remove any blood and preserve the tissue during preparation to mount the kidney on the isolated kidney perfusion set-up. The ureter was cannulated with a urinary catheter (Charriere 8) to allow urine collection during isolated kidney perfusion. Subsequently the abdominal aorta was punctured to collect 500 ml into a heparinised (3 ml, Heparine Leo®, Leo Pharma) collection bag, without any other additives.

### 1.3. Experimental Groups

Three experimental conditions were investigated: (a) warm ischaemia (WI) simulating anoxic/hypoxic acute kidney injury; (b) cold ischaemia (CI) replicating clinical cold storage preservation of kidney grafts for transplantation; and (c) controls. To obtain WI, kidneys were exposed to 60 minutes of ischaemia at body temperature (38° C., normothermia in the pig) by clamping the renal artery and vein before procurement. Kidneys were exposed to 22 hours of CI by submerging them in IGL-1 solution in a sealed plastic bag and storing them on ice. Control kidneys were procured as described above (“Pig experiments”) and immediately mounted on the isolated perfusion circuit.

### 1.4. Setting Up the Normothermic Isolated Kidney Perfusion Model

In a series of pilot experiments, pig kidneys were used to set up a model of normothermic isolated kidney perfusion (NIKP). Kidneys were perfused with a whole blood based perfusate at an arterial pressure of 60-70 mmHg at 38° C. for 4 hours. Pulsatile perfusion was achieved by a roller pump and a custom-made perfusion circuit including a membrane oxygenator and a heat exchanger. Kidneys (n=6) were retrieved after midline laparotomy, flushed with preservation solution (IGL-1) and prepared for NIKP by securing an arterial canula in the renal artery and a urine catheter in the ureter. Clinical read-outs were measured over 4 hours of NIKP and showed low vascular resistance, stable kidney function (creatinine clearance) and minimal cellular injury (AST) at the end of perfusion. We then perfused warm ischemically injured kidneys (n=2), exposed to 60 minutes of warm ischaemia (in situ clamping of renal artery and vein) before flushing them with IGL-1 and mounting on the NIKP circuit. These kidneys showed higher resistances with lower urine production and creatinine clearance and higher AST and h-FABP concentrations.

### 1.5. Normothermic Isolated Kidney Perfusion Set-Up

During normothermic isolated kidney perfusion (NIKP), perfusate is pumped through the kidney vasculature at a pre-set perfusion pressure at 38° C. (normothermia in pigs). The perfusate, running through non-heparin coated polyvinylchloride tubing (Intersept® Class VI measures ¼× 1/16, Medtronic, Belgium), is pushed forward by a roller pump (Stöckert, Germany) from a reservoir to a membrane oxygenator (Affinity Pixie with Cortiva Bioactive Surface (heparin coated), Medtronic, Belgium), where the perfusate is oxygenated (air flow 100 ml/min O₂, FiO₂ 21%), to the arterial cannula. The perfusate freely drains back into the reservoir as the renal vein is not cannulated, making this an open drainage system avoiding the risk of outflow obstruction. An external heat-exchanger (40° C.) is connected to the oxygenator warming the perfusate by counter current principle. Perfusate flow is measured by a flow probe (SonoTT Ultrasonic Flowcomputer, em-tec MEDICAL, Germany) positioned on the arterial line. An arterial pressure line is connected to the inflow cannula to monitor perfusion pressures. These are kept between 60-70 mmHg by manually adjusting the flow. Kidneys are perfused on NIKP for 4 hours.

### 1.6. Perfusate Composition

The circuit was primed with a crystalloid (Ringers solution; Table 2Error! Reference source not found.) and a colloid (Human Albumin 20%, CAF-DCF, Belgium). The circulating perfusate volume (580 ml) was kept constant by replacing urine volume by crystalloid infusion (Ringers solution) in a 1:1 fashion. In addition to heparin, creatinine (to monitor kidney function) and nutrients (glucose and glutamine) were added to the circuit as a bolus at the start and a continuous infusion of heparin, epoprostenol (Flolan®, GlaxoSmithKline), glucose and glutamine was started (Table 2). We aimed at a starting pH of 7.4 with addition of sodium bicarbonate when needed.

Red blood cells were used as oxygen carriers and the perfusate composition was aimed at an hematocrit of 20%. Two different types of perfusate were used: (a) a red cell based perfusate and (b) a whole blood based perfusate (Table 1).

TABLE 1
Composition of perfusates

| | |
|---|---|
| RBC perfusate | whole blood perfusate |
| pig kidneys | pig kidneys |

Priming solution

| RBC/Whole blood | ~150 | mL | ~230 | mL |
|---|---|---|---|---|
| Ringer | 335 | ml | 292 | mL |
| Perfusate Additives | | | | |
| Bolus | | | | |
| albumin 20% | 93 | mL | 58 | mL |
| creatinine | 145 | mg | 145 | mg |
| heparin | 1250 | IU | 1250 | IU |

| | | |
|---|---|---|
| glucose | 540 mg (5 mM) | 270 mg (2.58 mM) |
| glutamine | 84 mg (1 mM) | 42 mg (0.5 mM) |

Infusions

| | | | | |
|---|---|---|---|---|
| glucose | 80 | mg/h | 80 | mg/h |
| glutamine | 24 | mg/h | 24 | mg/h |
| heparin | 50 | IU/h | 50 | IU/h |
| epoprostenol | 8.3 | μg/h | 8.3 | μg/h |

The RBC perfusate is corrected for the loss of e.g. albumin, glucose and glutamine relative to the whole blood perfusate.

TABLE 2
Composition Ringers solution

| Compound | Concentration |
|---|---|
| NaCl | 8.6 g/L |
| KCl | 0.3 g/L |
| CaCl₂ | 0.33 g/L |

Hydrochloric acid and/or sodium hydroxide may be added for pH adjustment

### 1.7. Red Blood Cell Based Perfusate

In pigs, whole blood was collected as described above and washed with 0.9% NaCl (Autolog autotransfusion system, Medtronic, Belgium) and either used immediately or stored at 4° C. until usage.

### 1.8. Whole Blood Based Perfusate

In pigs, whole blood was collected as described above and either used immediately or stored at 4° C. until usage.

### 1.9. Sample Collection

Arterial perfusate samples were collected via a sample line on the arterial cannule, venous perfusate samples were collected from the outflow directly at the renal vein. Perfusate samples were collected in EDTA tubes, at baseline (before the kidney was mounted), every 15 minutes during the first hour and hourly thereafter. Arterial blood and venous blood gas values were determined at the same time intervals. Every hour, the urine produced by the kidneys was removed from the urine collector and transferred to urine tubes. One milliliter aliquots of perfusate and urine (centrifuged 3500 rpm, 10 minutes, 4° C.) were snap frozen in liquid nitrogen and stored at −80° C. until analysis.

For purpose of monitoring of analytes in the perfusate, the perfusate composition at the time point 15 minutes after start of the kidney perfusion was taken as reference point. At this time point, the wash-out fraction of the perfused kidney (causing variable initial changes in composition of the perfusion solution and obscuring interpretation of changes of individual analytes in the perfusate) has sufficiently mixed with the perfusate solution.

### 1.10. Clinical Read-Outs

From start of perfusion, hemodynamic parameters (pressure and flow) were determined every 15 minutes during the first hour and hourly thereafter. Kidney function during perfusion was assessed by adding a bolus of 145 mg creatinine at start and by monitoring the disappearance of creatinine in the perfusate throughout the perfusion. Creatinine was determined at the hospital's central laboratory (Enzymatic-creatininase peroxidase method, COBAS 800 Hitachi/Roche). To assess cellular injury, aspartate transaminase (AST) was measured in the perfusate and determined in the central laboratory (IFCC method, limit of detection 4 IU/L, Hitachi/Roche COBAS). PaO₂ and PvO₂ values were determined by blood gas analyzer (ABL800 Flex, Radiometer). Oxygen uptake was calculated as follows: flow (ml/min)×(PaO2−PvO2)/kidney weight (g). Enzyme-linked immunosorbent assay was used for human fatty acid binding protein (FABP; HK414, LOD391 μg/ml, Hycult, Biotech, Uden, the Netherlands) as a marker of distal tubular injury (Jochmans et al. 2011, J Ann Surg 254:784-91) and villin-1 (Decuypere et al. 2017, Transplantation 101:e330-e336). All clinical read-outs were corrected for kidney weight except Villin-1 as results were semi-quantitative.

### 1.11. Sample Preparation and Metabolomics Analyses

Samples were extracted in a 80% methanol (80:20 methanol:water)(methanol≥99.9%, HiPerSolv CHROMANORM®, ULTRA for LC-MS, suitable for UPLC/UHPLC-MS instruments, VWR, Belgium) extraction buffer containing 1 μM of deuterated (D27) myristic acid, D12 glucose, ¹³C5-D5-15N glutamic acid and D7-1⁵N4-arginine as internal standards. Practically, 10 μl of sample (perfusate) was added to 990 μl of the extraction buffer and stored overnight at −80° C. For urine analyses 50 μl was added to 450 μl of the extraction buffer. Insolubilities and precipitated proteins were removed by centrifugation at 20.000 g, for 15 min at 4° C. 200 μL of the supernatant was transferred to an appropriate mass-spectrometry vial. 10 μl was loaded onto an Ultra Performance Liquid Chromatograph equipped with a HILIC column (InfinityLab Poroshell 120 HILIC-Z PEEK-lined 2.1×150 mm, 2.7 μm column (Agilent)) and connected in-line to a Q-exactive Orbitrap Focus (Thermo Scientific) mass spectrometer. A linear gradient was built up starting with 90% solvent A (LC-MS grade acetonitrile, acetonitrile hypergrade for LC-MS LiChrosolv, Supelco (Merck), Germany) and 10% solvent B (10 mM ammonium acetate pH 9.3). At 2 min the gradient increased to 60% of solvent B and maintained at 60% until 15 min. The gradient returned to 10% solvent B at 16 min and remained until 25 min. The flow rate was 250 μl/min and the column was kept at 25° C. throughout the analysis. The mass spectrometer operated in negative ion mode, with a spray voltage of 2.9 kV and a temperature of the capillary of 325° C. Gas settings were as follows: sheat gas 40 and auxilary gas 15. The vaporizer temperature was set at 300° C. A full scan (resolution of 70.000 and scan range of m/z 70-1050) was applied. XCalibur version to operate the LC-MS was 4.2.47.

### 1.12. Data Processing

Metabolomics data analyses were performed with El_Maven version 0.12.0. Raw files were converted into mzML using the MSConvert option of Proteowizard (version 3.0.20247). Metabolites were identified using an in-house library containing exact mass and retention time. The mass accuracy was set at 10 ppm. The output files (.csv) were processed with the software Polly (Elucidata®, USA) for the correction of natural abundances and the fractional contribution calculations (based on Fernandez et al. 1996, J Mass Spectrom 31:255-62).

Graphs for clinical read-outs were made with Graphad Prism version 8.2.1 and presented with mean and standard deviation. Metabolite abundancies were presented as fold change in the boxplots made with IBM SPSS statistics version 27. Heatmaps were generated with the help of Metaboanalyst 5.0 where data only underwent log transformation before being integrated.

### 2. Results

### 2.1. A Clinically Relevant Model Distinguishing Post-Reperfusion Ischemic Injury

In a stable porcine model of normothermic isolated kidney perfusion (NIKP; schematically represented in FIG. 1), three clinically relevant conditions were investigated: (a) 22 h cold ischaemia (CI) replicating cold storage preservation of kidney grafts for transplantation; (b) 1 h warm ischaemia (WI) simulating anoxic/hypoxic acute kidney injury and (c) controls (C). Isolated kidneys were reperfused with a balanced red blood cell (RBC) based perfusate ensuring the kidney is fully biochemically active. “On-pump” kidney behaviour over the 4 h perfusion period was distinctly different between the three groups as reflected by the statistically significant different clinical read-outs. In order to confirm the biochemical relevance of RBC perfusion, kidneys were reperfused with whole blood in parallel, more closely mimicking in vivo physiology at reperfusion. Here as well, clinical read-outs were statistically significant across the conditions, except for diuresis (p=0.15). The comparable clinical read-outs of the kidneys in the RBC and whole blood model pointed towards a similar physiological kidney behaviour.

### 2.2. A Unique Nutritional Behaviour in Ischemically Damaged Kidneys

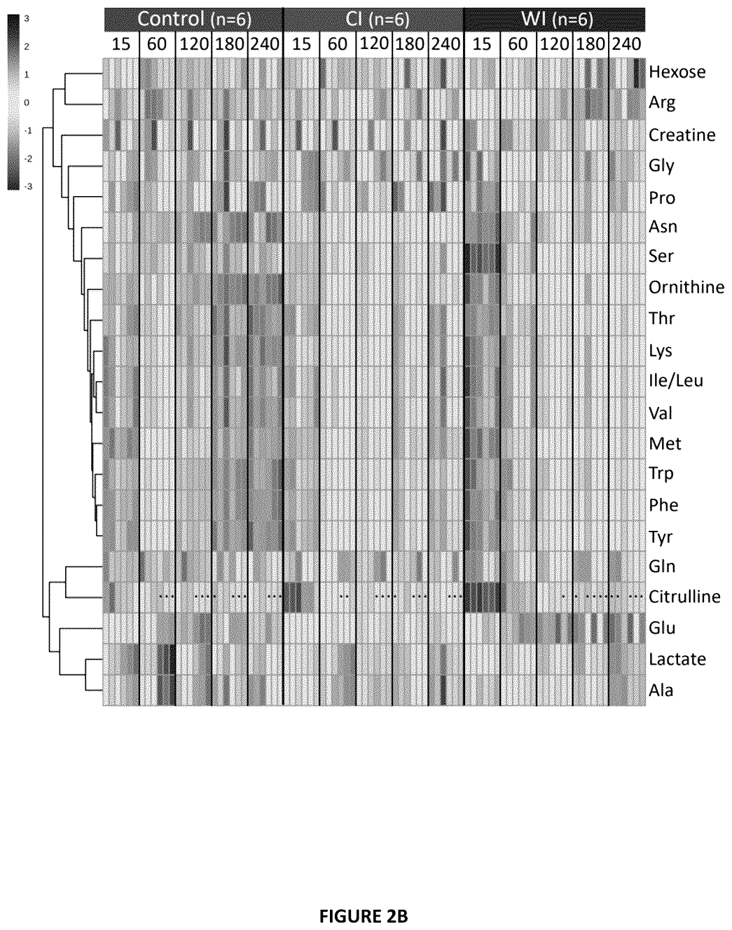

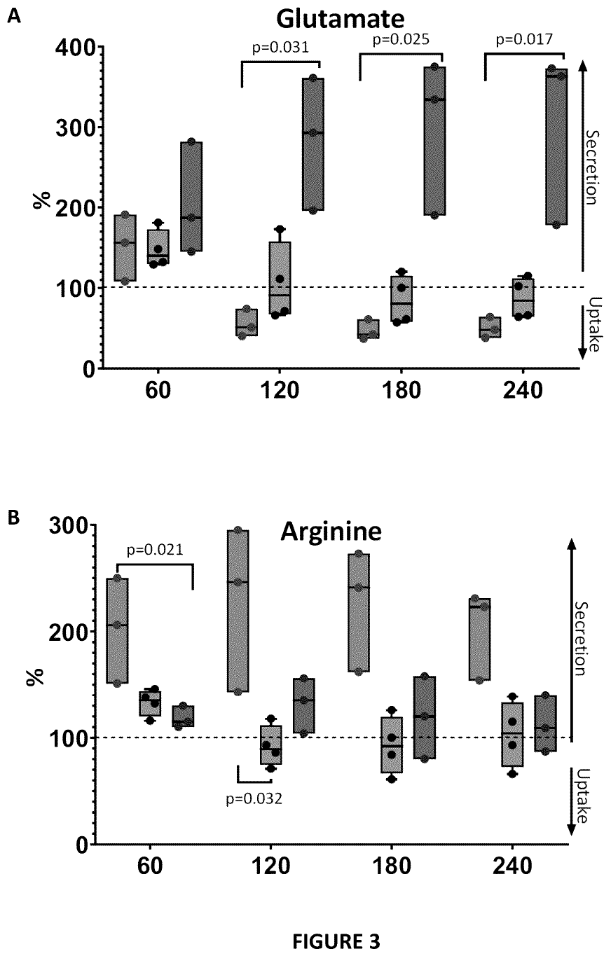

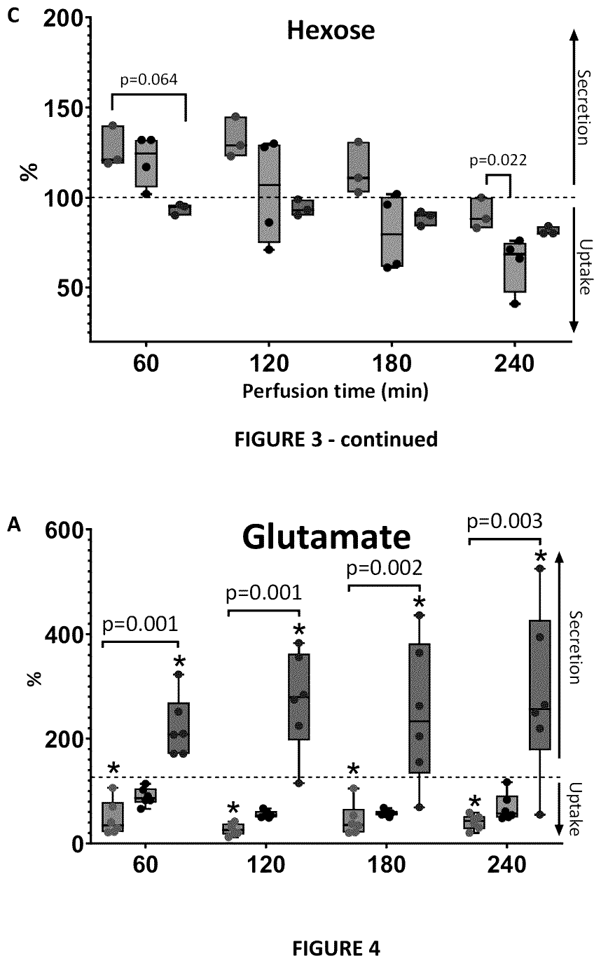

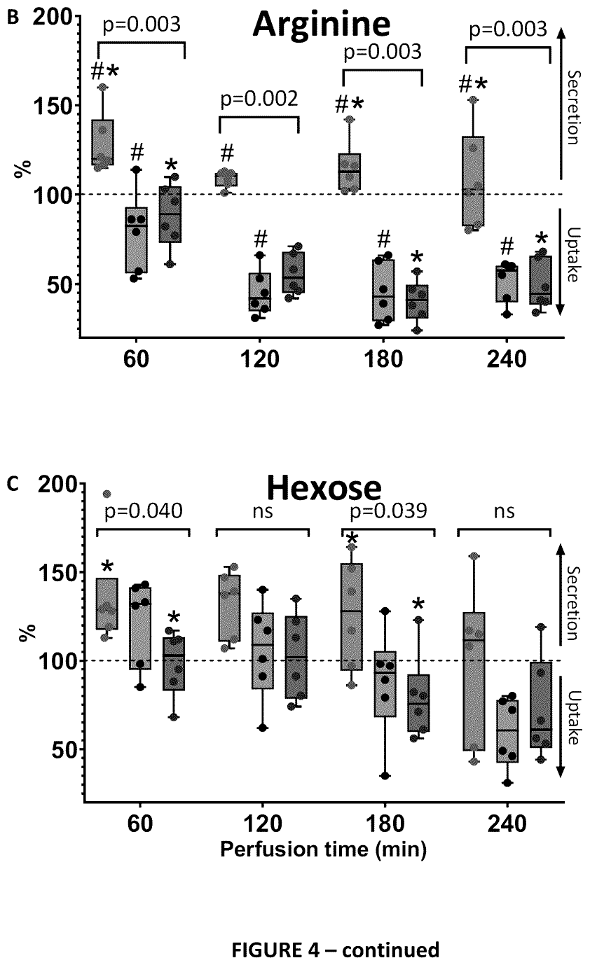

During kidney perfusion, a window of opportunity is created to assess their viability. We hypothesized that, during perfusion, changes in the uptake and secretion of metabolites would be altered amongst the three clinically relevant conditions studied. For this, perfusates were sampled during the 4-hour NIKP and analysed by LC-MS. We performed a partial least squares discriminant analysis (PLS-DA analysis; Metaboscape) from the quantified metabolites and could confirm clustering of the three conditions based on the relative changes of metabolite abundances in the perfusate. This difference was most pronounced at 2 hours in the RBC-perfused set-up, which is comparable to the commonly used clinical perfusate. Whole blood perfusion is a complex perfusate where variations in metabolite abundances influence the ability to find suitable ‘clear cut’ biomarkers. While the RBC perfusate is still ‘complex’, its composition is much more standardised and therefore provides a cleaner starting point to monitor differences in the biochemical behaviour of the perfused kidneys. Interestingly, the most significant differences between the ischemic (cold and warm) and control perfused kidneys occurred early during NIKP. This is an important asset for clinical applications where the ability to identify the kidney's viability at an early phase during the perfusion is vital. We also noticed that longer NIKP of the kidneys (>3 hours) seems to affect the metabolic behaviour and shifts control and cold ischemic kidneys towards the more detrimental warm ischemic profile. The heat maps (FIG. 2) reveal the changes of the quantified metabolites from the different conditions (C, CI and WI) in both RBC perfusate (FIG. 2A) and whole blood (FIG. 2B) over the course of 4 hours. Control (C) kidneys display an active metabolism and secrete several amino acids into the perfusate (threonine, lysine, valine, tryptophan, phenylalanine, tyrosine, etc). Also, several targets were found to be taken up as nutrients (citrulline, glutamine, and glutamate). Warm ischemically injured kidneys display a distinct profile where, in contrast to the control ‘healthy’ kidneys, a clear consumption of glucose and a secretion of glutamate was observed. Kidneys that underwent cold ischemia showed an intermediate profile that seems to share uptake and secretion profiles from the WI and C setups. From our perfusate analysis, 3 biomarkers of interest were identified that allowed distinguishing between the different conditions (FIGS. 3 and 4): glutamate was solely secreted by WI kidneys, arginine was solely consumed by ischemic (warm and cold) kidneys and glucose (hexose) was produced by the control kidneys during the first 2 hours of NIKP. The changes of these analytes/markers were observed in both RBC (FIG. 3) and whole blood (FIG. 4) perfused kidneys.

The heat map data were converted to quantitative data (except for arginine, glucose and glutamate as these are depicted in FIGS. 3 and 4), normalized to the 15 min post-perfusion reference data and expressed as % (as in FIGS. 3 and 4). These quantitative data are provided in Table 3 (RBC-based perfusate) and Table 4 (WBC-based perfusate). From these, further analytes/markers of interest were derived: glutamine, lactate, and alanine. The levels of glutamine in the perfusate of a non-ischemic perfused kidney are decreasing. The decrease in levels or concentrations of glutamine are less significant in cold ischemic kidneys. The decrease in levels or concentrations of glutamine are less significant in warm ischemic kidneys compared to cold ischemic kidneys and healthy kidneys. The levels of lactate in the perfusate of kidneys are significantly increasing in non-ischemic kidneys. The increase in levels or concentrations of lactate is less significant in the perfusate of warm ischemic kidneys compared to healthy kidneys. The increase in levels or concentrations of lactate is less significant in the perfusate of cold ischemic kidneys compared to warm ischemic kidneys and healthy kidneys. The levels of alanine in the perfusate of kidneys are increasing in non-ischemic kidneys, in cold ischemic kidneys and in warm ischemic kidneys. The increase is more pronounced in warm ischemic kidneys compared to healthy kidneys. The increase is less pronounced in cold ischemic kidneys compared to healthy kidneys. The levels of leucine in the perfusate of kidneys are increasing in all kidneys. The increase is, however, more significant for non-ischemic kidneys compared to both warm and cold ischemic kidneys. The levels of isoleucine in the perfusate of kidneys are increasing in all kidneys. The increase is, however, more significant for non-ischemic kidneys compared to both warm and cold ischemic kidneys.

TABLE 3
Analysis of changes over time (minutes; versus the reference
point determined at 15 min after start of the perfusion;
60, 120, 180 and 240 min) in the amounts of the indicated
analyte in the perfusate of pig kidneys perfused ex-vivo
with a red blood cell (RBC)-based perfusate.

| | 60 | | | 120 | | |
|---|---|---|---|---|---|---|
| | CTRL | WI | CI | CTRL | WI | CI |
| lactate | 73% | 188% | 79% | 213% | 287% | 137% |
| glycine | 184% | 177% | 147% | 231% | 239% | 168% |
| isoleucine/leucine | 338% | 166% | 222% | 522% | 249% | 286% |
| alanine | 67% | 190% | 88% | 193% | 315% | 111% |
| asparagine | 407% | 249% | 250% | 641% | 439% | 272% |
| glutamine | 82% | 81% | 65% | 41% | 60% | 38% |
| lysine | 261% | 125% | 185% | 444% | 199% | 261% |
| methionine | 416% | 227% | 271% | 765% | 398% | 414% |
| phenylalanine | 380% | 176% | 246% | 637% | 281% | 351% |
| proline | 263% | 191% | 205% | 327% | 300% | 230% |
| serine | 328% | 303% | 259% | 419% | 614% | 306% |
| threonine | 329% | 186% | 232% | 524% | 293% | 306% |
| tryptophane | 370% | 186% | 255% | 712% | 293% | 408% |
| tyrosine | 370% | 191% | 254% | 637% | 308% | 360% |
| valine | 297% | 149% | 206% | 469% | 209% | 279% |
| creatine | 164% | 175% | 146% | 220% | 230% | 184% |
| ornithine | 292% | 173% | 194% | 492% | 270% | 252% |
| | 180 | | | 240 | | |
| | CTRL | WI | CI | CTRL | WI | CI |
| lactate | 467% | 365% | 290% | 660% | 492% | 441% |
| glycine | 244% | 264% | 186% | 256% | 283% | 210% |
| isoleucine/leucine | 602% | 301% | 342% | 633% | 344% | 404% |
| alanine | 301% | 420% | 184% | 366% | 537% | 269% |
| asparagine | 580% | 567% | 274% | 544% | 598% | 275% |
| glutamine | 14% | 45% | 34% | 13% | 47% | 32% |
| lysine | 551% | 257% | 316% | 613% | 298% | 378% |
| methionine | 948% | 529% | 543% | 1086% | 633% | 656% |
| phenylalanine | 771% | 361% | 429% | 854% | 425% | 510% |
| proline | 339% | 367% | 264% | 354% | 420% | 317% |
| serine | 398% | 837% | 306% | 396% | 949% | 321% |
| threonine | 611% | 373% | 360% | 679% | 424% | 428% |
| tryptophane | 856% | 359% | 470% | 925% | 420% | 516% |
| tyrosine | 800% | 392% | 439% | 915% | 452% | 513% |
| valine | 555% | 251% | 334% | 605% | 283% | 390% |
| creatine | 261% | 243% | 198% | 284% | 256% | 211% |
| ornithine | 618% | 331% | 272% | 672% | 366% | 300% |

CTRL: non-ischemic kidneys; WI: warm ischemic kidneys; CI: cold ischemic conditions.

TABLE 4
Analysis of changes over time (minutes; versus the reference
point determined at 15 min after start of the perfusion;
60, 120, 180 and 240 min) in the amounts of the indicated
analyte in the perfusate of pig kidneys perfused ex-vivo
with a whole blood (WBC)-based perfusate.

| | 60 | | | 120 | | |
|---|---|---|---|---|---|---|
| | CTRL | WI | CI | CTRL | WI | CI |
| lactate | 96% | 192% | 128% | 182% | 234% | 126% |
| glycine | 166% | 185% | 136% | 155% | 211% | 118% |
| isoleucine/leucine | 218% | 162% | 179% | 251% | 210% | 186% |
| alanine | 110% | 171% | 137% | 133% | 207% | 128% |
| asparagine | 300% | 154% | 211% | 336% | 181% | 187% |
| glutamine | 56% | 77% | 35% | 29% | 52% | 23% |
| lysine | 222% | 151% | 169% | 292% | 196% | 186% |
| methionine | 320% | 238% | 247% | 459% | 382% | 312% |
| phenylalanine | 292% | 169% | 222% | 392% | 241% | 263% |
| proline | 192% | 161% | 170% | 194% | 204% | 170% |
| serine | 331% | 353% | 314% | 364% | 659% | 329% |
| threonine | 274% | 155% | 261% | 363% | 217% | 323% |
| tryptophane | 259% | 162% | 219% | 367% | 226% | 272% |
| tyrosine | 306% | 200% | 252% | 445% | 300% | 317% |
| valine | 213% | 149% | 170% | 259% | 193% | 183% |
| creatine | 141% | 118% | 122% | 144% | 134% | 113% |
| citrulline | 15% | 35% | 10% | 12% | 6% | 5% |
| ornithine | 254% | 202% | 207% | 333% | 263% | 217% |
| | 180 | | | 240 | | |
| | CTRL | WI | CI | CTRL | WI | CI |
| lactate | 440% | 247% | 184% | 501% | 409% | 257% |
| glycine | 177% | 183% | 122% | 148% | 246% | 144% |
| isoleucine/leucine | 324% | 202% | 230% | 339% | 287% | 257% |
| alanine | 239% | 208% | 168% | 269% | 367% | 232% |
| asparagine | 359% | 156% | 203% | 318% | 196% | 180% |
| glutamine | 28% | 57% | 23% | 19% | 66% | 27% |
| lysine | 386% | 192% | 236% | 383% | 273% | 270% |
| methionine | 668% | 396% | 421% | 708% | 613% | 497% |
| phenylalanine | 539% | 234% | 342% | 560% | 364% | 387% |
| proline | 247% | 182% | 202% | 243% | 274% | 233% |
| serine | 386% | 656% | 347% | 339% | 938% | 386% |
| threonine | 492% | 219% | 427% | 517% | 336% | 480% |
| tryptophane | 503% | 226% | 335% | 483% | 335% | 345% |
| tyrosine | 648% | 303% | 418% | 707% | 468% | 468% |
| valine | 350% | 187% | 229% | 365% | 270% | 255% |
| creatine | 172% | 125% | 127% | 164% | 175% | 130% |
| citrulline | 8% | 3% | 4% | 12% | 2% | 5% |
| ornithine | 448% | 226% | 237% | 428% | 309% | 246% |

CTRL: non-ischemic kidneys; WI: warm ischemic kidneys; CI: cold ischemic conditions.

This points towards a unique and early window of opportunity during NIKP to assess the viability of kidneys by monitoring the nutritional behaviour and secretion of metabolic products in addition to the established clinical parameters.

Moreover, since these profiles are similar between the RBC and whole blood, evidencing that RBC perfusion closely mimics an in vivo situation, we decided to continue with the RBC-based NIKP because the metabolic variations found in the whole blood perfusate were less pronounced and the biochemical functions of the kidney were similar between RBC and whole blood.

### 2.3. Nutritional Behaviour of Perfused Kidneys and Secretion of Metabolic Products by Perfused Kidneys is Predictive of Future Kidney Function

Kidney function can be assessed by measuring clearance of creatinine from the perfusate over time. Some exploratory work looked into the link between changes in levels or concentrations in the perfusate of glutamate, arginine, and glucose and changes in creatinine levels or concentrations in the perfusate. A liner regression model for the relative change of creatinine concentration at 4 h was constructed in which the kidney condition (Control, WI, CI) and the metabolite of interest were entered as covariates.

These models showed that glutamate and arginine were independent predictors of kidney function as assessed by the relative change in creatinine concentration, and this independent of the kidney condition.

The exploratory analyses suggest that clustering is best when assessing at 2 h. Furthermore, as an earlier time point during perfusion is of more interest to clinicians as this facilitates decision making processes and transplant logistics, we explored whether relative changes in abundance of glutamate, arginine, and glucose at 2 h of perfusion were predictive of kidney function as assessed by relative changes in creatinine concentration at 4 h of perfusion. Here we found that all 3 metabolites were independent predictors of kidney function at the end of perfusion.

### Example 3. Analysis of Human Kidney Clinical Perfusate Samples

In the study reported by Rijkse et al. 2021 (BJS Open 5(1), zraa024; POSEIDON study, MEC 2017-503), kidneys were preserved by hypothermic perfusion (HP) followed by 2 hours of normothermic perfusion (NP) and then transplanted. Kidneys were either DBD (donation after brain death; n=4) or DCD (donation after circulatory death, n=7). DCD kidneys experienced a period of warm ischemia before start of HP. During the study, perfusate samples were collected at predetermined time points (at the end of hypothermic perfusion (HP) and at baseline, 30 min, 1 h, 2 h during normothermic perfusion (NP)). Urine samples were collected as well at predetermined time points.

Kidneys were perfused with KPS-1 (www.organ-recovery.com/preservation-solutions/kps-1-kidney-perfusion-solution/) during HP. Perfusion during NP was with a red blood cell-based solution (see Table 1 of Rijkse et al. 2021, BJS Open 5(1), zraa024; with the exception that the Olimel N7E amino acid/glucose mixture was used instead of the Nutriflex® infusion; the Olimel N7E composition is provided in Table 5), in a continuous infusion (25 ml/h). Origin of the baseline perfusate sample (time 0) is not clear as it may have been taken before or after the start of the infusion. The exact target concentrations at the start of perfusion cannot be retrieved as the perfusate volume was not collected. In a further difference with the conditions as outlined in Examples 1 and 2, urine was recirculated, which might have influenced the metabolite concentrations. Of note, in the conditions outlined in Examples 1 and 2, only glucose and glutamine were added and urine was diverted from the circuit and replaced by Ringers solution (crystalloid solution).

TABLE 5
Olimel N7E composition

| Compartment | Content per 1000 ml |
|---|---|
| 35% glucose solution (35 g/100 mL) | 400 mL |
| 11.1% amino acid solution (11.1 g/100 mL) | 400 mL |
| 20% lipid emulsion (20 g/100 mL) | 200 mL |

Composition of the reconstituted emulsion after mixing the content of the 3 compartments:

| Active substances | 1000 mL |
|---|---|
| Refined olive oil + refined soybean oil* | 40.00 g |
| Alanine | 6.41 g |
| Arginine | 4.34 g |
| Aspartic acid | 1.28 g |
| Glutamic acid | 2.21 g |
| Glycine | 3.07 g |
| Histidine | 2.64 g |
| Isoleucine | 2.21 g |
| Leucine | 3.07 g |
| Lysine | 3.48 g |
| (equivalent to lysine acetate) | (4.88 g) |
| Methionine | 2.21 g |
| Phenylalanine | 3.07 g |
| Proline | 2.64 g |
| Serine | 1.75 g |
| Threonine | 2.21 g |
| Tryptophan | 0.74 g |
| Tyrosine | 0.11 g |
| Valine | 2.83 g |
| Sodium acetate, trihydrate | 1.50 g |
| Sodium glycerophosphate, hydrated | 3.67 g |
| Potassium chloride | 2.24 g |
| Magnesium chloride, hexahydrate | 0.81 g |
| Calcium chloride, dihydrate | 0.52 g |
| Glucose anhydrous | 140.00 g |
| (equivalent to glucose monohydrate) | (154.00 g) |

*mixture of refined olive oil (approximately 80%) and refined soybean oil (approximately 20%) corresponding to a ratio essential fatty acids/total fatty acids of 20%.

Perfusate and urine samples were collected as indicated above. Furthermore, post-transplant outcome behavior of the donor kidneys is available and was defined as:
immediate post-transplant outcome (delayed graft function, i.e. the need for dialysis in the first week after transplantation; duration of delayed graft function; primary non function, i.e. a never functioning graft, diagnosed retrospectively at 3 months post-transplantation)
acute rejection (within 12 months, proven by biopsy)
1-year graft survival.

Other post-transplant outcomes of donor kidney function included measurement of (a) estimated glomerular filtration rate (eGFR) (CKD-EPI) or of (b) serum creatinine collected at 3, 6 and 12 months post-transplantation. eGFR is expressed in ml/min/1.73 m\hat{ }2 (ml of blood filtered by the kidneys per minute, corrected for the patient's body surface area). Renal filtration rate is calculated with the CKD-EPI (Chronic Kidney Disease Epidemiology Collaboration) formula, considered as being the most reliable, based on the amount of creatinine in the blood corrected for age, gender and race.

Metabolomic profiles of the perfusate and urine samples collected as part of this study were generated. These profiles include but are not limited to carbohydrates, amino acids, lipids and other metabolites. Samples were prepared for and analysed on an established platforms to identify metabolite changes during perfusion. One of several mass spectrometry (MS) platforms (Liquid Chromatography (LC) (HILIC, C18, ion-pairing, etc) and Gas Chromatography (GC) based Mass Spectrometers (OrbiTRAP, QQQ, MR-MS, etc) were used. A combination of in-house developed software pipelines and commercial software (Polly, Elucidata) for the processing of the raw MS generated data files was/is used.

A full perfusate and urinary metabolomic profile of human kidneys undergoing normothermic perfusion was obtained. It was/is established whether any of the metabolites emerges as candidate markers predictive of post-transplant outcome/kidney function. It was/is also investigated whether the metabolic profile or components of it correlate with other markers measured during perfusion (e.g. perfusion characteristics such as flow, pH, etc.).

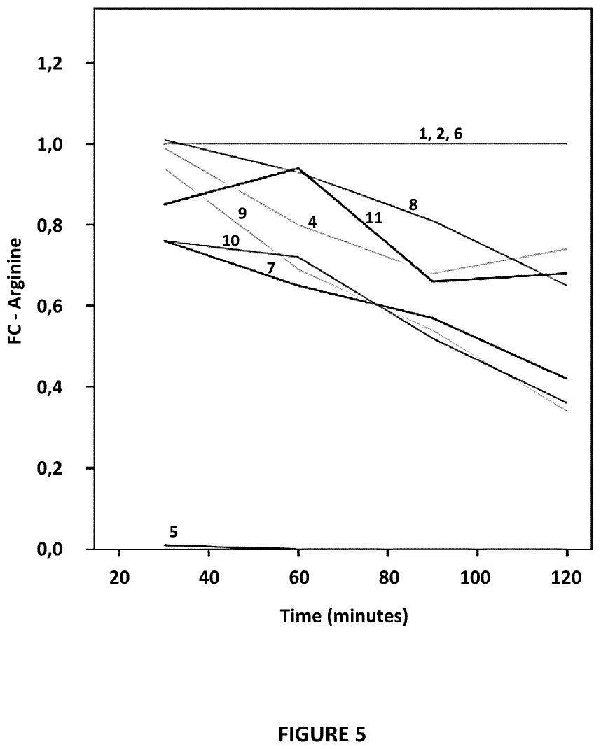

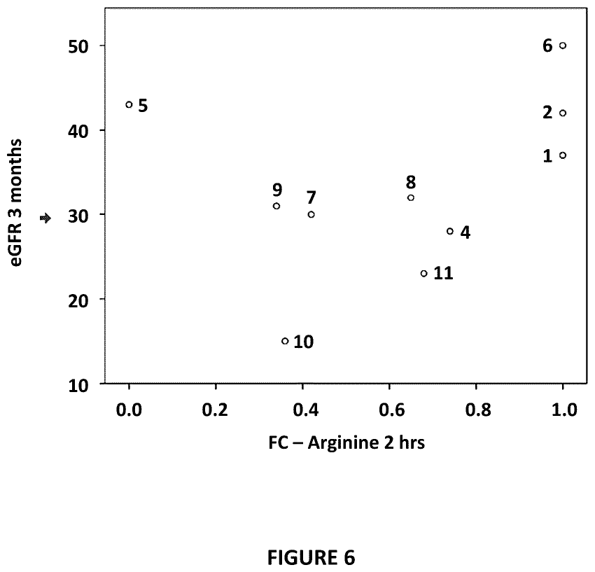

With the cautionary notes (i) that the clinical conditions in this study are not comparable with the more controlled experimental conditions as outline in Example 1 and 2, and (ii) that the analyses of the metabolomic profiles are as yet incomplete, some preliminary outcomes of these analyses focused on the metabolite arginine and are included here:
overall metabolite concentrations at baseline varied considerably—a plausible explanation probably being the inconsistent composition of the perfusion solution nutrients at baseline (see higher)
due to missing time points for 1 kidney, this one (kidney number 3) was excluded from the analysis
arginine was not detectable in the perfusate of 3 kidneys (kidneys number 1, 2, and 6); kidney number 5 is reminiscent of this group with the difference that arginine was measurable at t=0 but disappeared by the t=30 min time point; the reason for this behavior of these kidneys (numbers 1, 2, 6, and 5) being different from the other kidneys (kidney numbers 4, 7, 8, 9, 10, and 11) is as yet unclear
longitudinal changes as a fold change (FC) compared to time 0 (arginine concentration at time x divided by arginine concentration at time 0) were tracked and shown as spaghetti plot in FIG. 5; in this figure the FC values of 3 kidneys with undetectable arginine in the perfusate all equal 1 (no change, i.e. constant non-detectable); a group of kidneys (kidney numbers 4, 7, 8, 9, 10, and 11) behaved similarly in that they showed a decrease of perfusate arginine, possibly indicative of the presence of ischemic damage (cfr. observations with perfused pig kidneys in Example 2.2 hereinabove; with the caveat that such possible ischemia in the human kidneys, if present, would have occurred in a less controlled way compared to the pig kidneys)
within the group of kidneys behaving similarly (kidney numbers 4, 7, 8, 9, 10, and 11; the kidneys that showed a decrease of perfusate arginine, see FIG. 5), a correlation might exist between arginine levels in the perfusate and eGFR at 3 months with the lower the relative change in arginine (FC nearer to 1; kidney numbers 4, 8, and 11) the better the kidney function (higher eGFR), as shown in FIG. 6. Such correlation is reminiscent of the observations made in Example 2.3 hereinabove (with the caveat that for the pig kidneys no post-transplantation functional data were available). The 3 kidneys with an FC of 1 (arginine constantly non-detectable), and kidney number 5, had a higher than median/mean eGFR values (median 31 ml/min/1.73 m²; mean 32.3 ml/min/1.73 m²).

These preliminary data provide the first indication, for the metabolite arginine, that the observations as made in the more controlled setup of pig kidney perfusion may be found in perfused human kidneys and that there may be a correlation between arginine depletion during the pre-transplantation perfusion period and the post-transplant functioning of the human kidney. An additional observation from this clinical setting is that human kidneys perfused under these conditions and characterized by the absence of detectable arginine (kidneys 1, 2 and 6) or very rapid clearing of residual arginine (kidney 5) appear to function well after transplantation, such as characterized by a higher than average eGFR at 3 months.

Similar analyses are performed for the ongoing APOLLO study (ClinicalTrials.gov identifier NCT04882254). In this study, kidneys are preserved by hypothermic perfusion (HP) or hypothermic oxygenated perfusion (HOPE) followed by 2 hours of normothermic perfusion (NP) and then transplanted. This study includes BDB kidneys (combination of HP and NP) and DCD kidneys (HP+NP; or HOPE+NP; both preceded by period of warm ischemia).

1. A method of analyzing the perfusate of a kidney which is ex vivo or ex situ perfused, comprising:
sampling the perfusate during or after initiating the perfusion;
determining the presence, level or concentration of the analyte arginine in the perfusate sample(s).

2. The method according to claim 1 which is a method of determining the presence, absence or severity of ischemic damage in a kidney and is further comprising:
determining ischemic damage to be present in the perfused kidney when the determined presence, level or concentration of the analyte arginine is deviating or is significantly deviating from the control or reference level or concentration of the analyte arginine; or, alternatively, determining ischemic damage to be absent in the perfused kidney when the determined level or concentration of the analyte arginine is not or not significantly deviating from the control or reference level or concentration of the analyte arginine; or, alternatively, determining the severity of ischemic damage in the perfused organ from the extent of deviation of the determined level or concentration of the analyte arginine from the control or reference level or concentration of the analyte arginine.

3. A method of therapeutic optimization of, repair of, or reconditioning of a kidney prior to transplantation, comprising one or more steps of:
connecting the kidney to an ex vivo or ex situ perfusion device;
performing the therapeutic optimization, repair or reconditioning of the perfused kidney during perfusion;
sampling the perfusate during or after initiating the perfusion;
determining the presence, level or concentration of the analyte arginine in the perfusate sample(s).

4. A method of developing or optimizing kidney perfusion solutions or kidney preservation solutions, or of optimizing kidney perfusion conditions, comprising:
connecting the kidney to an ex vivo or ex situ perfusion device;
perfusing the kidney with a test perfusion or preservation solution, or perfusing the kidney under test perfusion conditions;
monitoring the presence, level or concentration of the analyte arginine in the perfusate.

5. A method of screening for modalities capable of reversing or partially reversing defects of an kidney; or of screening for modalities capable of reversing or partially reversing ischemic damage in a kidney; or of screening for modalities capable of improving or enhancing kidney function, comprising one or more steps of:
connecting the kidney to an ex vivo or ex situ perfusion device;
adding to the perfusion solution a test modality, wherein the test modality is designed to reverse or to partially reverse a defect of or in a kidney; or wherein the test modality is designed to reverse or to partially reverse ischemic damage in a kidney; or wherein the test modality is designed to improve or enhance kidney function;
monitoring the presence, level or concentration of the analyte arginine in the perfusate.

6. The method according to any of the foregoing claims further comprising determining the presence, level or concentration one or more of the analytes glutamate, glutamine, glucose, lactate, alanine, leucine and/or isoleucine in the perfusate sample(s);

7. The method according to claim 6 wherein:
when the presence, level or concentration of glutamate is determined, the presence, level or concentration of at least 1 or more of the analytes arginine, glutamine, glucose, lactate, alanine, leucine and/or isoleucine is determined; or
when the presence, level or concentration of glucose is determined, the presence, level or concentration of at least 1 or more of the analytes arginine, glutamate, glutamine, alanine and/or isoleucine is determined; or
when the presence, level or concentration of lactate is determined, the presence, level or concentration of at least 1 or more of the analytes arginine, glutamate, glutamine, alanine, leucine and/or isoleucine is determined; or
when the presence, level or concentration of leucine is determined, the presence, level or concentration of at least 1 or more of the analytes arginine, glutamate, glutamine, lactate, alanine, and/or isoleucine is determined.

8. A machine perfusion apparatus or device, an organ transporter, or an organ cassette, further characterized in that it is comprising a unit or module for determining the presence, level or concentration of the analyte arginine in the perfusate of an organ ex situ perfused with a perfusion solution.

9. A method of analyzing the perfusate of an organ perfused ex vivo or ex situ with a perfusion solution, comprising:
sampling the perfusate at different time points during the ex vivo or ex situ perfusion;
determining a reference time point which is the time point during perfusion at which the organ wash-out fraction has homogenized with the initial perfusion solution; and, optionally,
setting the presence, level or concentration of an analyte of interest determined in the perfusate at the reference time point as reference or control level or concentration of the analyte of interest.
